(19) United States
(12) Patent Application Publication
(10) Pub. No.: US 2007/0208112 A1
(43) Pub. Date: Sep. 6, 2007

(54) Use of Pyridindione Derivatives for Protecting Organic Material Against Detrimental Effects of Light

(75) Inventors: Simon Schambony, Ludwigshafen (DE); Alban Glaser, Mannheim (DE); Rudiger Sens, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/592,666

(22) PCT Filed: Apr. 14, 2005
(86) PCT No.: PCT/EP05/03917

§ 371 Date: Sep. 13, 2006

(30) Foreign Application Priority Data
Apr. 16, 2004 (DE) 10 2004 019 171.9

(51) Int. Cl. C08K 5/34; C07D 211/78; C07D 211/72

(52) U.S. Cl. 524/99; 546/268.1; 546/288; 546/296

(57) ABSTRACT

The present invention relates to the use of pyridinedione derivatives of general formula I 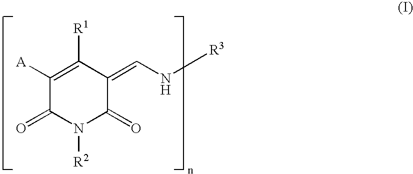 and if appropriate their tautomers in which
R¹ is hydrogen, optionally substituted and/or if appropriate heteroatom-comprising alkyl, alkenyl or alkynyl or optionally substituted cycloalkyl, cycloalkenyl, heterocycloalkyl, aryl or heteroaryl,
R² independently of R¹ has the definition of R¹ or NR⁴R⁵,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹, n denotes values of 1, 2, 3 or 4,
R³ if n is 1: is hydrogen, optionally substituted and/or if appropriate heteroatom-comprising alkyl, alkenyl or alkynyl or optionally substituted cycloalkyl, cycloalkenyl or heterocycloalkyl, if n is not 1: is an n-valent aliphatic or cycloaliphatic radical which may if appropriate comprise heteroatoms, to protect organic material from the damaging effects of light, to compositions which comprise at least one such pyridinedione derivative of formula I in an amount conferring protection from the damaging effects of light, and at least one organic material, and to pyridinedione derivatives of formula I.

The present invention relates to the use of pyridinedione derivatives for protecting organic material from the damaging effects of light, to compositions which comprise at least one such pyridinedione derivative in an amount conferring protection from the damaging effects of light, and at least one organic material and to such pyridinedione derivatives.

Living and inanimate organic material such as human or animal skin, human or animal hair, paper, foods, perfumes, cosmetics, plastics, polymer dispersions, paints, photographic emulsions, photographic layers, etc., is frequently sensitive to the damaging effects of light, and particularly to the ultraviolet (UV) radiation fraction which is present in daylight. The damage may be due to the UVA fraction of the UV radiation, i.e., the region from above 320 to 400 nm, the UVB fraction of UV radiation, i.e. the region from 280 to 320 nm, and the even shorter-wave fractions of UV radiation.

Said damage is normally manifested as yellowing, discoloration, cracking or embrittlement in the material. One important field of use for light stabilizers is therefore the protection of plastics. Plastic containers and plastic films find widespread use as packaging materials, for example. For reasons of esthetics, plastics featuring high light transmission in the visible wavelength range from 400 to 750 nm are increasingly gaining importance as packaging materials. See-through plastics with or without slight coloration, however, are generally transparent to the UV fractions of daylight, with the consequence that, under light, both the packaging material and the packaged products suffer aging. Depending on the specific packaged contents, the adverse alteration to the contents may be manifested, for example, in a change in appearance, such as yellowing and discoloration, in a change in taste and/or odor, and/or in the breakdown of ingredients. In the case of foods, perfumes, and cosmetics, the shelf life and keeping properties may be sharply reduced. The stabilizers that are added to packaging plastic ought therefore to provide satisfactory protection both to the plastic itself and to the packaged product in respect of light-induced aging processes. Plastics are also in widespread use in combination with glass in composite materials which are transparent in the visible wavelength range. Composite systems of this kind find use in automotive and architectural glazing, for example. Automobile windows of this kind are increasingly being required to absorb radiation in the wavelength range below 400 nm, in order to protect the interior of the auto and its occupants against UV radiation, for example.

The prior art light stabilizers have a series of disadvantages. One substantial disadvantage is the period of the protective effect, which is often too short, since the known light stabilizers often have an inadequate UV stability. Another disadvantage is that many known light stabilizers have a clearly perceptible intrinsic color in the visible wavelength range, with the consequence that a plastic stabilized with these light stabilizers appears to have a pale yellow coloration. Furthermore, many light stabilizers are unable to filter out the longwave fraction of the UVA radiation and/or have too low a radiation absorption efficiency, i.e., their extinction coefficient is too low. Many light stabilizers, moreover, lack adequate solubility in the application medium. The resulting crystallization of the light stabilizer may make the polymer opaque. Further disadvantages are the frequently poor synthetic obtainability of the light stabilizers, their inadequate formulating properties, their low sublimation resistance and/or their low migration fastness

Pyridinedione derivatives are already known as dyes and dye precursors.

Thus the publication by F. Würthner, Synthesis 1999, No 12, 2104-2113 concerns itself with the synthesis of merocyanine dyes, among which mention is made of 5-phenylaminomethylene-1-butyl-4-methyl-3-cyano-2,6-dioxo-1,2,5,6-tetrahydropyridine.

The publication WO 03/063151 A2 gives a description, among other things, of the use of tautomeric pyridinediones of formulae 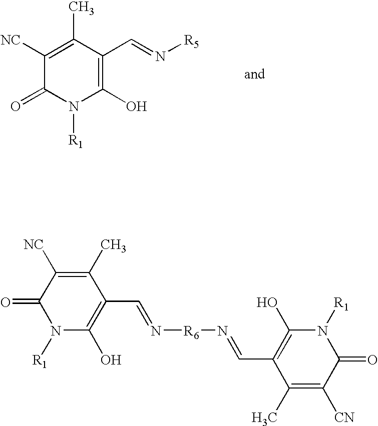
in optical recording layers, the radicals R₅ and R₆, according to Examples 1 to 50 disclosed in said publication, consistently being monovalent or divalent aromatic or heteroaromatic groups, and R₁ being an ethyl radical.

The use of pyridinedione derivatives to protect organic material from the damaging effects of light, on the other hand, was hitherto unknown in the state of the art.

In view of the continuing high demand for stabilizers and stabilizer compositions it was an object of the present invention to provide stabilizers which are suitable as UV absorbers for protecting organic material such as plastics, polymer dispersions, paints, photographic emulsions, photographic layers, paper, human or animal skin, human or animal hair, foods, etc. for a relatively long period of time. The stabilizers should preferably absorb with high extinction in the UVA region, and particularly also in the longwave UVA region above 360 nm, ought to be photostable and/or thermally stable, and ought to have little or no intrinsic color in the visible wavelength range. The stabilizers ought also to have advantageous performance properties and/or to be easy to prepare.

Surprisingly it has now been found that the use of pyridinedione derivatives of general formula I 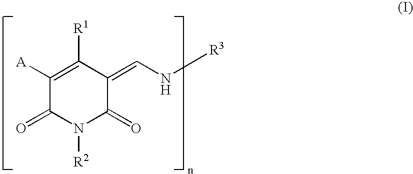
and if appropriate their tautomers
in which
R¹ is hydrogen, optionally substituted and/or if appropriate heteroatom-comprising alkyl, alkenyl or alkynyl or optionally substituted cycloalkyl, cycloalkenyl, heterocycloalkyl, aryl or heteroaryl,
R² independently of R¹ has the definition of R¹ or NR⁴R⁵,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹,
n denotes values of 1, 2, 3 or 4,
R³ if n is 1:
   is hydrogen, optionally substituted and/or if appropriate heteroatom-comprising alkyl, alkenyl or alkynyl or optionally substituted cycloalkyl, cycloalkenyl or heterocycloalkyl,
   if n is not 1:
   is an n-valent aliphatic or cycloaliphatic radical which may if appropriate comprise heteroatoms,
      protects organic material against the damaging effects of light.

Alkyl comprises straight-chain or branched alkyl, such as for example methyl, ethyl, propyl, isopropyl, n-butyl, isobutyl, sec-butyl, tert-butyl, n-pentyl, 2-pentyl, 2-methyl-butyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-di-methylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, 1,1,3,3-tetramethylbutyl, nonyl, decyl, n-undecyl, n-dodecyl, n-tridecyl, isotridecyl, n-tetradecyl, n-hexadecyl, n-octadecyl and n-eicosyl.

Alkyl also comprises alkyl whose carbon chain may be interrupted by one or more nonadjacent groups selected from —O—, —S—, —NZ¹— or —NZ⁵—, —CO— and —SO₂—, i.e., the alkyl groups are terminated by carbon atoms. Z¹ and Z⁵ respectively here are hydrogen, C₁-C₁₈ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

Alkenyl comprises straight-chain and branched alkenyl groups which depending on the chain length may carry one or more double bonds. Preference is given to C₂-C₂₀, more preferably C₂-C₁₀, alkenyl groups, such as vinyl, allyl or methallyl. Alkenyl also comprises substituted alkenyl groups, which for example may carry one, two, three, four or five substitutents. Examples of suitable substitutents are cycloalkyl, heterocycloalkyl, aryl, heteroaryl, nitro, cyano, halogen, amino or mono- or di-(C₁-C₂₀ alkyl)amino.

Alkynyl comprises straight-chain and branched alkynyl groups, which depending on the chain length may carry one or more triple bonds. Preference is given to C₂-C₂₀, more preferably C₂-C₁₀, alkynyl groups, such as ethynyl, propyn-3-yl or propyn-1-yl. Alkynyl also comprises substituted alkynyl groups, which for example may carry one, two, three, four or five substitutents. Examples of suitable substitutents are cycloalkyl, heterocycloalkyl, aryl, heteroaryl, nitro, cyano, halogen, amino or mono- or di-(C₁-C₂₀ alkyl)amino.

Cycloalkyl comprises both unsubstituted and substituted cycloalkyl groups, preferably C₅-C₈ cycloalkyl groups, such as cyclopentyl, cyclohexyl, cycloheptyl or cyclooctyl. In the case of a substitution the cycloalkyl groups may carry one or more—for example, one, two, three, four or five—C₁-C₆ alkyl groups.

Examples of C₅-C₈ cycloalkyl, which is unsubstituted or carries one or more C₁-C₆ alkyl groups, are cyclopentyl, 1-, 2- and 3-methylcyclopentyl, 1-, 2- and 3-ethylcyclopentyl, cyclohexyl, 1-, 2-, 3- and 4-methylcyclohexyl, 1-, 2-, 3- and 4-ethylcyclohexyl, 1-, 3- and 4-propylcyclohexyl, 1-, 3- and 4-isopropylcyclohexyl, 1-, 3- and 4-butylcyclohexyl, 1-, 3- and 4-sec-butylcyclohexyl, 1-, 3- and 4-tert-butylcyclohexyl, cycloheptyl, 1-, 2-, 3- and 4-methylcycloheptyl, 1-, 2-, 3- and 4-ethylcycloheptyl, 1-, 3- and 4-propylcycloheptyl, 1-, 3- and 4-isopropylcycloheptyl, 1-, 3- and 4-butylcycloheptyl, 1-, 3- and 4-sec-butyl-cycloheptyl, 1-, 3- and 4-tert-butylcycloheptyl, cyclooctyl, 1-, 2-, 3-, 4- and 5-methyl-cyclooctyl, 1-, 2-, 3-, 4- and 5-ethylcyclooctyl and 1-, 3-, 4- and 5-propylcyclooctyl.

Cycloalkenyl comprises both unsubstituted and substituted cycloalkenyl groups, preferably C₅-C₈-cycloalkenyl groups, such as 1-cyclopenten-1-yl, 1-cyclohexen-1-yl, 1-cyclohepten-1-yl and 1-cycloocten-1-yl and the radicals that are positionally isomeric thereto, namely 1-cyclopenten-3-yl, 1-cyclopenten-4-yl, 1-cyclohexen-3-yl, 1-cyclohexen-4-yl, 1-cyclohepten-3-yl, 1-cyclohepten-4-yl, 1-cyclohepten-5-yl, 1-cycloocten-3-yl, 1-cycloocten-4-yl and 1-cycloocten-5-yl. In the case of a substitution the cycloalkenyl groups may carry one or more—for example, one, two, three, four or five—C₁-C₆ alkyl groups.

Heterocycloalkyl comprises nonaromatic, unsaturated or fully saturated, cycloaliphatic groups having in general five to eight ring atoms, preferably five or six ring atoms, in which one, two or three of the ring carbon atoms have been replaced by heteroatoms, selected from oxygen, sulfur, and the group —NZ—, and which is unsubstituted or substituted by one or more—for example, one, two, three, four, five or six—C₁-C₆ alkyl groups. Examples that may be mentioned of such heterocycloaliphatic groups include pyrrolidinyl, piperidyl, 2,2,6,6-tetramethylpiperidyl, especially 2,2,6,6-tetramethylpiperid-4-yl, imidazolidyl, pyrazolidyl, oxazolidyl, morpholyl, thiazolidyl, isothiazolidyl, isoxazolidyl, piperazinyl, tetrahydrothiophenyl, dihydrothienyl, tetrahydrofuranyl, dihydrofuranyl, tetrahydropyranyl, 1,2-oxazolinyl, 1,3-oxazolinyl and dioxanyl, Z in the —NZ— moiety corresponding in its definition to Z¹.

Aryl comprises monocyclic or polycyclic aromatic hydrocarbon radicals which can be unsubstituted or substituted, and is preferably phenyl, tolyl, xylyl, mesityl, duryl, naphthyl, fluorenyl, anthracenyl, phenanthrenyl or naphthyl, more preferably phenyl or naphthyl, it being possible for these aryl groups, in the case of a substitution, to carry generally one, two, three, four or five, preferably, one, two or three, substitutents.

Heteroaryl comprises unsubstituted or substituted, heteroaromatic, monocyclic or polycyclic groups, preferably the groups pyridyl, quinolyl, acridinyl, pyridazinyl, pyrimidyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, it being possible for these heterocycloaromatic groups, in the case of a substitution, to carry generally one, two or three substitutents.

n-Valent organic radicals include those radicals which derive formally from the aforementioned, optionally substituted and/or if appropriate heteroatom-comprising alkyl, alkenyl or alkynyl radicals or optionally substituted cycloalkyl, cycloalkenyl and heterocycloalkyl radicals by abstracting one (n=2), two (n=3) or three (n=4) further hydrogen atoms.

As a divalent, trivalent or tetravalent organic radical suitability is also possessed by a group of general formula: 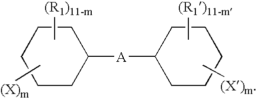
in which:
A is a single chemical bond, —O—, —S—, —NY—, —CO—, —SO₂—, C₁-C₁₈ alkylene whose carbon chain may be interrupted by one or more nonadjacent groups, selected from —O—, —S—, —NY′—, —CO— and —SO₂— and/or may be substituted by one or more C₁-C₆ alkyl radicals,
R₁ and R₁′ independently of one another are hydrogen, C₁-C₆ alkyl, C₁-C₆ alkoxy, hydroxyl, carboxyl or cyano,
X is a single chemical bond,
Y and Y′ independently of one another are hydrogen, C₁-C₆-alkyl, aryl or heteroaryl, it being possible for each of aryl and heteroaryl to be unsubstituted or to carry one or more substitutents selected from the group consisting of C₁-C₆ alkyl, C₁-C₆ alkoxy, hydroxyl, carboxyl and cyano,
   and
m and m′ denote values of 0, 1, 2, 3 or 4, with the proviso that m+m′ gives values of 2, 3 or 4.

Divalent organic radicals include:

C₂-C₃₀ alkylene whose carbon chain may be interrupted by one or more nonadjacent groups selected from —O—, —S—, —NZ⁶—, —CO— and —SO₂—, in particular by one or more nonadjacent groups selected from —O—, —NZ⁶—and —CO—, preferably C₂-C₁₂ alkylene whose carbon chain may be interrupted by one or more nonadjacent groups selected from —O— and —NZ⁶—, and more preferably C₂-C₁₂ alkyl,

C₅-C₈ cycloalkylene which is unsubstituted or carries one or more C₁-C₆ alkyl groups, in particular one or more C₁-C₄ alkyl groups, preferably cyclopentylene or cyclohexylene, each of which is unsubstituted or carries one or more C₁-C₄ alkyl groups, more preferably cyclopentylene or cyclohexylene, especially cis- or trans-cyclohexane-1,4-diyl,

5- to 8-membered heterocycloalkylene which is unsubstituted or carries one or more C₁-C₆ alkyl groups, in particular one or more C₁-C₄ alkyl groups, preferably divalent piperidyl which is unsubstituted or carries one or more C₁-C₄ alkyl groups,

Z⁶ being hydrogen, C₁-C₁₈ alkyl, in particular C₁-C₆ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

Trivalent or tetravalent organic radicals include:

trivalent or tetravalent C₂-C₃₀ alkyl whose carbon chain may be interrupted by one or more nonadjacent groups selected from —O—, —S—, —NZ⁶—, —CO— and —SO₂—, in particular by one or more nonadjacent groups selected from —O—, —NZ⁶—and —CO—, preferably trivalent or tetravalent C₂-C₁₂ alkyl whose carbon chain may be interrupted by one or more nonadjacent groups selected from —O— and —NZ⁶—, and more preferably trivalent or tetravalent C₂-C₁₂ alkyl,

trivalent or tetravalent C₅-C₈ cycloalkyl which is unsubstituted or carries one or more C₁-C₆ alkyl groups, in particular one or more C₁-C₄ alkyl groups, preferably trivalent or tetravalent cyclopentyl or trivalent or tetravalent cyclohexyl, each of which is unsubstituted or carries one or more C₁-C₄ alkyl groups, more preferably trivalent or tetravalent cyclopentyl or trivalent or tetravalent cyclohexyl,

trivalent or tetravalent 5- to 8-membered heterocycloalkyl which is unsubstituted or carries one or more C₁-C₆ alkyl groups, in particular one or more C₁-C₄ alkyl groups, preferably trivalent or tetravalent piperidyl which is unsubstituted or carries one or more C₁-C₄ alkyl groups,

Z⁶ being hydrogen, C₁-C₁₈ alkyl, in particular C₁-C₆ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

Preferred is the use of compounds of formula I wherein
R¹ is hydrogen,
   C₁-C₃₀ alkyl whose carbon chain may be interrupted by one or more nonadjacent groups selected from —O—, —S—, —NZ¹—, —CO— and —SO₂— and/or which is optionally substituted one or more times by identical or different radicals selected from the group consisting of cyano, amino, hydroxyl, halogen, carboxyl, C₁-C₁₈ alkoxycarbonyl, C₁-C₁₈ alkanoyloxy, aryl, heterocycloalkyl and heteroaryl, with the aryl, heterocycloalkyl, and heteroaryl groups being unsubstituted or carrying one or more substitutents selected independently of one another from the group consisting of C₁-C₁₈ alkyl and C₁-C₆ alkoxy,
   C₅-C₈ cycloalkyl or 5- to 8-membered heterocycloalkyl, which are unsubstituted or carry one or more C₁-C₆ alkyl groups,
   or
   aryl or heteroaryl which are unsubstituted or carry one or more radicals selected independently of one another from the group consisting of C₁-C₁₈ alkyl, C₁-C₆ alkoxy, cyano, CONZ²Z³, CO₂Z⁴,
R² independently of R¹ has the definition of R¹ or NR⁴R⁵,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹,
n denotes values of 1, 2, 3 or 4
   and
R³ if n is 1:
   is hydrogen,
   C₁-C₃₀ alkyl whose carbon chain may be interrupted by one or more nonadjacent groups selected from —O—, —S—, —NZ⁵—, —CO— and —SO₂— and/or which is optionally substituted one or more times by identical or different radicals selected from the group consisting of cyano, amino, hydroxyl, halogen, carboxyl, C₁-C₁₈ alkoxycarbonyl, C₁-C₁₈ alkanoyloxy, aryl, heterocycloalkyl and heteroaryl, with the aryl, heterocycloalkyl, and heteroaryl groups being unsubstituted or carrying one or more substitutents selected independently of one another from the group consisting of C₁-C₁₈ alkyl and C₁-C₆ alkoxy,
   or
   C₅-C₈ cycloalkyl or 5- to 8-membered heterocycloalkyl, which are unsubstituted or carry one or more C₁-C₆ alkyl groups,
   if n is not 1:
   n-valent C₂-C₃₀ alkyl whose carbon chain may be interrupted by one or more nonadjacent groups selected from —O—, —S—, —NZ⁶—, —CO— and —SO₂—,
   or
   n-valent C₅-C₈ cycloalkyl or n-valent 5- to 8-membered heterocycloalkyl, which are unsubstituted or carry one or more C₁-C₆ alkyl groups,
   Z¹, Z², Z³, Z⁴, Z⁵ and Z⁶ each independently of one another being hydrogen, C₁-C₁₈ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

Suitable C₁-C₁₈ alkyl and C₁-C₆ alkyl radicals as substitutents have already been set out by way of example above under the alkyl radicals. C₁-C₆ Alkoxy radicals and the C₁-C₁₈ alkoxy radicals of C₁-C₁₈ alkoxycarbonyl derive formally from the corresponding alkyl radicals by addition of a terminal oxygen atom. The C₁-C₁₈ alkanoyl radicals of the C₁-C₁₈ alkanoyloxy radicals are obtained formally by replacing a terminal methylene group with the corresponding alkyl radical by a carbonyl group.

Examples of aryl which is unsubstituted or carries one or more mutually independent C₁-C₁₈ alkyl, C₁-C₆ alkoxy or cyano radicals are 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl, 2-, 3- and 4-butoxyphenyl; 2-, 3- and 4-cyanophenyl.

In another preferred embodiment the use is preferred of compounds of formula I wherein
R¹ is hydrogen,
   C₁-C₁₅ alkyl whose carbon chain may be interrupted by one or more nonadjacent groups selected from —O—, —NZ¹— and —CO— and/or which is optionally substituted one or more times by identical or different radicals selected from the group consisting of cyano, amino, hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₆ alkyl and C₁-C₆ alkoxy,
   cyclopentyl or cyclohexyl, which are unsubstituted or carry one or more C₁-C₆ alkyl groups,
   piperidyl which is unsubstituted or carries one or more C₁-C₆ alkyl groups
   or
   phenyl which is unsubstituted or carries one or more radicals selected independently of one another from the group consisting of C₁-C₆ alkyl, C₁-C₆ alkoxy, cyano, CONZ²Z³, CO₂Z⁴,
R² independently of R¹ has the definition of R¹ or NR⁴R⁵,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹,
n denotes values of 1, 2, 3 or 4
   and
R³ if n is 1:
   is hydrogen,
   C₁-C₁₅ alkyl whose carbon chain may be interrupted by one or more nonadjacent groups selected from —O—, —NZ⁵—and —CO— and/or which is optionally substituted one or more times by identical or different radicals selected from the group consisting of cyano, amino, hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₆ alkyl and C₁-C₆ alkoxy,
   cyclopentyl or cyclohexyl, which are unsubstituted or carry one or more C₁-C₆ alkyl groups,
   or
   piperidyl which is unsubstituted or carries one or more C₁-C₆ alkyl groups,
   if n is not 1:
   n-valent C₂-C₃₀ alkyl whose carbon chain may be interrupted by one or more nonadjacent groups selected from —O—, —NZ⁶—and —CO—,
   or
   n-valent C₅-C₈ cycloalkyl or n-valent 5- to 8-membered heterocycloalkyl, which are unsubstituted or carry one or more C₁-C₄ alkyl groups,
   Z¹, Z², Z³, Z⁴, Z⁵ and Z⁶ each independently of one another being hydrogen, C₁-C₁₈ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

In another preferred embodiment it is preferred to use compounds of formula I wherein
R¹ is hydrogen,
   C₁-C₈ alkyl which is optionally substituted one or more times by identical or different radicals selected from the group consisting of halogen and aryl, with the aryl group being unsubstituted or carrying one or more substitutents selected independently of one another from the group consisting of C₁-C₄ alkyl and C₁-C₄ alkoxy, cyclopentyl or cyclohexyl, which are unsubstituted or carry one or more C₁-C₄ alkyl groups,
   piperidyl which is unsubstituted or carries one or more C₁-C₄ alkyl groups
   or
   phenyl which is unsubstituted or carries one or more radicals selected independently of one another from the group consisting of C₁-C₄ alkyl, C₁-C₄ alkoxy and cyano,
R² independently of R¹ has the definition of R¹ or NR⁴R⁵,
R⁴, R⁵ independently of one another and of R¹ have the definition of R¹ or COR⁶,
A is CN,
R⁶ independently of R¹ has the definition of R¹,
n denotes values of 1, 2, 3 or 4
   and
R³ if n is 1:
   is hydrogen,
   C₁-C₈ alkyl whose carbon chain may be interrupted by one or more nonadjacent groups selected from —O—, —NZ⁵—and —CO— and/or which is optionally substituted one or more times by identical or different radicals selected from the group consisting of hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₄ alkyl and C₁-C₄ alkoxy;
   cyclopentyl or cyclohexyl, which are unsubstituted or carry one or more C₁-C₄ alkyl groups,
   or
   piperidyl which is unsubstituted or carries one or more C₁-C₄ alkyl groups, if n is not 1:
   n-valent C₂-C₁₂ alkyl whose carbon chain may be interrupted by one or more nonadjacent groups selected from —O— and —NZ⁶—,
   n-valent cyclopentyl or n-valent cyclohexyl, which are unsubstituted or carry one or more C₁-C₄ alkyl groups,
   or
   n-valent 5- to 8-membered heterocycloalkyl which is unsubstituted or carries one or more C₁-C₄ alkyl groups,
   Z⁵ and Z⁶ being hydrogen or C₁-C₆ alkyl.

In a further preferred embodiment preference is given to using compounds of formula I wherein
R¹ is hydrogen,
   C₁-C₄ alkyl, partly fluorinated or perfluorinated C₁-C₄ alkyl
   or
   phenyl which is unsubstituted or carries one or more radicals selected independently of one another from the group consisting of C₁-C₄ alkyl, C₁-C₄ alkoxy and cyano,
R² independently of R¹ has the definition of R¹ or NR⁴R⁵,
R⁴, R⁵ independently of one another and of R¹ have the definition of R¹ or COR⁶,
A is CN,
R⁶ independently of R¹ has the definition of R¹,
n denotes values of 1, 2, 3 or 4
   and
R³ if n is 1:
   is hydrogen,
   C₁-C₈ alkyl whose carbon chain may be interrupted by one group —O— and/or which is optionally substituted one or more times by identical or different radicals selected from the group consisting of hydroxyl, fluoro, carboxyl, C₁-C₄ alkoxycarbonyl, C₁-C₄ alkanoyloxy and aryl, with the aryl group being unsubstituted or carrying one or more substitutents selected independently of one another from the group consisting of C₁-C₄ alkyl and C₁-C₄ alkoxy,
   cyclopentyl or cyclohexyl, which are unsubstituted or carry one or more C₁-C₄ alkyl groups,
   or
   piperidyl which is unsubstituted or carries one or more C₁-C₄ alkyl groups,
   if n is not 1:
   n-valent C₂-C₁₂ alkyl, n-valent cyclopentyl, n-valent cyclohexyl or n-valent piperidyl, which is unsubstituted or carries one or more C₁-C₄ alkyl groups.

The compounds of formula I can be prepared in accordance with German laid-open specification DE 20 25 327 by reacting hydroxypyridones with dimethylformamide in accordance with the following equation 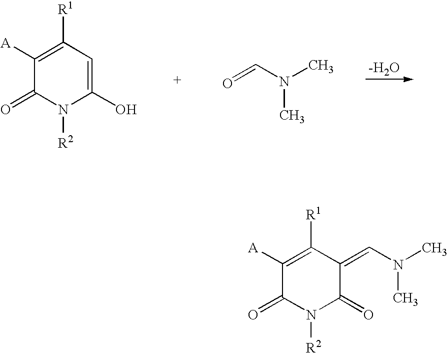
to give the 5-dimethylaminomethylene-substituted pyridinedione compounds and then reacting these compounds in accordance with specification WO 03/063151 A2 with the desired n-functional amines in accordance with the reaction equation 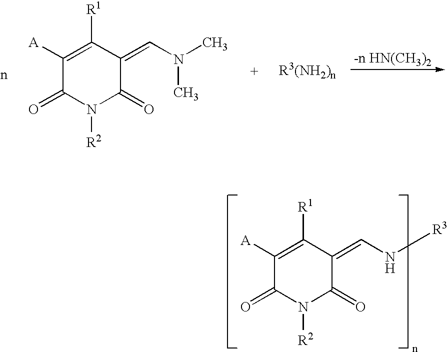
to give the target compounds.

The term “protection” of organic material is to be interpreted widely in the context of the present specification. It comprises on the one hand the stabilization of an organic material with respect to the damaging effects of light in order to prevent and/or at least retard light-initiated degradation processes in the organic material. For this purpose an agent which protects against light is added to the organic material. On the other hand the aforementioned term, in the context of the present specifics, further embraces the indirect protection of organic materials, where an organic material comprising an agent that protects against light surrounds at least partly another organic material, so as to reduce the damaging effects of light for the organic material behind.

The pyridinedione derivatives of formula I are used inventively to protect organic material from the damaging effects of light, organic material for the purposes of the present specification meaning not only living organic material but also inanimate organic material. Living organic material is for example human or animal skin; inanimate organic material is for example human or animal hair. Examples of inanimate organic material further include foods, cleaning products, perfumes, textiles, paper, furniture, carpets, plastic moldings such as electrical housings, cosmetic preparations such as ointments, creams, gels, emulsions and lotions, drug formulations such as drops, emulsions, solutions, pills, tablets and suppositories, paints, photographic emulsions, photographic layers, and particularly plastics and polymer dispersions.

It is preferred to use pyridinedione derivatives of the formula I to protect inanimate organic material, particularly to protect plastics. The pyridinedione derivatives of formula I are distinguished by high compatibility with plastics, so that the polymer's optical properties, as well as its other properties, are not impaired.

Preferred plastics and polymer blends are those which are transparent in the visible wavelength range in the uncolored state and which can be processed to highly transparent, glass-clear packs or packing materials. They include not only homopolymers and copolymers but also physical blends of polymers (polymer blends), copolymers being obtained by (joint) copolymerization of two or more different monomers. It will be appreciated that copolymers such as polyesters may also comprise transesterification products, depending on the preparation and/or processing method. In the context of the preparation and/or processing method it is also possible, in the case of copolymers, for grafting to occur, including transfer grafts.

The pyridinedione derivatives of formula I for inventive use not only protect plastics against the consequences of light exposure but also—especially in the case of transparent plastics—protect living and/or inanimate organic material at least partly surrounded by the plastic protected with the pyridinedione derivatives of the formula I against the damaging effects of light.

The present invention accordingly further provides for the use of at least one pyridinedione derivative of general formula I or one of its preferred embodiments for preparing a layer which absorbs ultraviolet light. The material of the layer is preferably composed of thermoplastic polymers which find use in particular as packaging materials or in sheet form.

As thermoplastic polymers mention may be made in particular of polycarbonates, polyesters, polyvinyl acetals, polyolefins, poly(meth)acrylates, polyacryinitrile, and polyvinyl chloride; and also of copolymers, obtained by copolymerizing the monomers on which said polymers are based, and also polymer blends comprising said polymers.

Suitable plastics comprise at least one polyester, preferably at least one linear polyester. Suitable polyesters and copolyesters are described in EP-A-0678376, EP-A-0 595 413 and U.S. Pat. No. 6,096,854, hereby incorporated by reference. Polyesters, as is known, are condensation products of one or more polyols and one or more polycarboxylic acids. In linear polyesters the polyol is a diol and the polycarboxylic acid is a dicarboxylic acid. The diol component may be selected from ethylene glycol, 1,4-cyclohexanedimethanol, 1,2-propanediol, 1,3-propanediol, 1,4-butanediol, 2,2-dimethyl-1,3-propanediol, 1,6-hexanediol, 1,2-cyclohexanediol, 1,4-cyclohexanediol, 1,2-cyclohexanedimethanol, and 1,3-cyclohexanedimethanol. Also suitable are diols whose alkylene chain is interrupted one or more times by nonadjacent oxygen atoms. Such diols include diethylene glycol, triethylene glycol, dipropylene glycol, tripropylene glycol, and the like. In general the diol comprises from 2 to 18 carbon atoms, preferably 2 to 8 carbon atoms. Cycloaliphatic diols can be used in the form of their cis or trans isomer or as an isomer mixture. The acid component can be an aliphatic, alicyclic or aromatic dicarboxylic acid. The acid component of linear polyesters is generally selected from terephthalic acid, isophthalic acid, 1,4-cyclohexanedicarboxylic acid, 1,3-cyclohexanedicarboxylic acid, succinic acid, glutaric acid, adipic acid, sebacic acid, 1,12-dodecanedioic acid, 2,6-naphthalenedicarboxylic acid, and mixtures thereof. It will be appreciated that the functional derivatives of the acid component can also be used, such as esters, the methyl ester for example, anhydrides or halides, preferably chlorides. Preferred polyesters are polyalkylene terephthalates and polyalkylene naphthalates obtainable by condensing terephthalic acid or naphthalenedicarboxylic acid, respectively, with an aliphatic diol.

Particularly preferred polyalkylene terephthalates are polyethylene terephthalates (PET), which are obtained by condensing terephthalic acid with ethylene glycol. PET is also obtainable by transesterifying dimethyl terephthalate with ethylene glycol, with elimination of methanol, to form bis(2-hydroxyethyl) terephthalate, and subjecting the product to polycondensation, releasing ethylene glycol. Further preferred polyesters are polybutylene terephthalates (PBT), obtainable by condensing terephthalic acid with 1,4-butanediol, polyethylene 2,6-naphthalate (PEN), poly-1,4-cyclohexanedimethylene terephthalates (PCT), and also copolyesters of polyethylene terephthalate with cyclohexanedimethanol (PDCT) and of polybutylene terephthalate with cyclohexanedimethanol. Preference is likewise given to copolymers, transesterification products, and physical mixtures (blends) of the aforementioned polyalkylene terephthalates. Particularly suitable thermoplastic molding compounds are selected from polycondensates and copolycondensates of terephthalic acid, such as poly- or copolyethylene terephthalate (PET or CoPET or PETG), poly(ethylene 2,6-naphthalate)s (PEN) or PEN/PET copolymers and PEN/PET blends. Said copolymers and blends, depending on their preparation process, may also comprise fractions of transesterification products.

Further suitable plastics comprise at least one polycarbonate polymer, selected from the group consisting of polycarbonates, polycarbonate copolymers and physical blends based on polycarbonates with acrylic-butadiene-styrene copolymers, acrylonitrile-styrene-acrylate copolymers, polymethyl methacrylates, polybutyl acrylates, polybutyl methacrylates, poly(butylene terephthalate)s and polyethylene terephthalates.

Polycarbonates are formed, for example, by condensation of phosgene or carbonic esters such as diphenyl carbonate or dimethyl carbonate with dihydroxy compounds.

Suitable dihydroxy compounds are aliphatic or aromatic dihydroxy compounds. Examples of aromatic dihydroxy compounds include bisphenols such as 2,2-bis(4-hydroxyphenyl)propane (bisphenol A), tetraalkylbisphenol A, 4,4-(meta-phenylenediisopropyl)diphenol (bisphenol M), 4,4-(para-phenylenediisopropyl)diphenol, 1,1-bis(4-hydroxyphenyl)-3,3,5-trimethylcyclohexane (BP-TMC), 2,2-bis(4-hydroxyphenyl)-2-phenylethane, 1,1-bis(4-hydroxyphenyl)cyclohexane (bisphenol Z) and, if appropriate, mixtures thereof. The polycarbonates may be branched by using small amounts of branching agents. Suitable branching agents include phloroglucinol, 4,6-dimethyl-2,4,6-tri(4-hydroxyphenyl)hept-2-ene, 4,6-dimethyl-2,4,6-tri(4-hydroxyphenyl)heptane; 1,3,5-tri(4-hydroxyphenyl)benzene; 1,1,1-tri(4-hydroxyphenyl)heptane; 1,3,5-tri(4-hydroxyphenyl)benzene; 1,1,1-tri(4-hydroxyphenyl)-ethane; tri(4-hydroxyphenyl)phenylmethane, 2,2-bis[4,4-bis(4-hydroxyphenyl)-cyclohexyl]propane; 2,4-bis(4-hydroxyphenylisopropyl)phenol; 2,6-bis(2-hydroxy-5′-methylbenzyl)-4-methylphenol; 2-(4-hydroxyphenyl)-2-(2,4-dihydroxyphenyl)propane; hexa(4-(4-hydroxyphenylisopropyl)phenyl)orthoterephthalate; tetra(4-hydroxyphenyl)-methane; tetra(4-(4-hydroxyphenylisopropyl)phenoxy)methane; a,a′,a″-tris(4-hydroxyphenyl)-1,3,5-triisopropylbenzene; 2,4-dihydroxybenzoic acid; trimesic acid; cyanuric chloride; 3,3-bis(3-methyl-4-hydroxyphenyl)-2-oxo-2,3-dihydroindole, 1,4-bis(4′,4″-dihydroxytriphenyl)methyl)benzene, and, in particular, 1,1,1-tri(4-hydroxyphenyl)ethane and bis(3-methyl-4-hydroxyphenyl)-2-oxo-2,3-dihydroindole.

For chain termination, suitability is possessed by, for example, phenols such as phenol, alkylphenols such as cresol and 4-tert-butylphenol, chlorophenol, bromophenol, cumylphenol or mixtures thereof. The fraction of chain terminators is generally from 1 to 20 mol %, per mole of dihydroxy compound.

Further suitable plastics comprise at least one polymer derived from α,β-unsaturated acids and derivatives thereof, e.g., poly(meth)acrylate such as polymethyl methacrylate (PMMA) and polyethyl methacrylate.

Further suitable plastics comprise at least one vinylaromatic homopolymer or copolymer such as polystyrene (PS) or a copolymer of styrene or α-methylstyrene with dienes and/or acrylic derivatives, such as styrene-butadiene, styrene-acrylonitrile (SAN), styrene-ethyl methacrylate, styrene-butadiene-ethyl acrylate, styrene-acrylonitrile-methacrylate, acrylonitrile-butadiene-styrene (ABS) or methyl methacrylate-butadiene-styrene (MBS).

Further suitable plastics comprise at least one polymer derived from unsaturated alcohols and amines or from their acyl derivatives or acetals, such as polyvinyl acetate (PVAC), polyvinyl alcohol (PVAL), polyvinyl formyl (PVFM) or polyvinyl butyral (PVB). Polyvinyl acetals are obtainable by reacting polyvinyl alcohol with aldehydes. The two last-mentioned compounds, PVFM and PVB, can be prepared correspondingly by reacting polyvinyl alcohol with formaldehyde or with butyraldehyde.

In laminated glass, two or more sheets of glass are bonded together adhesively through polyvinyl butyral films. The polyvinyl butyral molding compound generally has an average molecular mass of more than 70 000, preferably from about 100 000 to 250 000. The polyvinyl butyral generally has a residual hydroxyl group content of less than 19.5%, preferably from about 17% to 19% by weight, calculated as polyvinyl alcohol, and a residual ester group content of from 0 to 10%, preferably from 0 to 3%, calculated as polyvinyl ester. An exemplary PVB is obtainable commercially under the name Butvar® from Solutia, Inc. of St. Louis, Mo. Any glass is suitable provided it is transparent for light in the visible wavelength range. Such glasses comprise normal clear soda-lime glass, IR-reflecting coated glass or IR-absorbing glass; see, e.g., U.S. Pat. No. 3,944,352 and U.S. Pat. No. 3,652,303. With respect to the configuration of laminated glass, the entirety of WO 02/077081, and in particular pages 28 to 32, is hereby incorporated by reference.

Further suitable plastics are polyolefins which comprise all polymers synthesized from olefins without further functionality, such as low or high density polyethylene, polypropylene, linear polybut-1-ene or polyisobutylene or polybutadiene, and also copolymers of monoolefins or diolefins. Preferred polyolefins are the homopolymers and copolymers of ethylene and also the homopolymers and copolymers of propylene.

Ethylene Polymers:

Suitable polyethylene (PE) homopolymers are, for example:
PE-LD (LD=low density), obtainable for example by the high-pressure process (ICI) at 1000 to 3000 bar and 150 to 300° C. with oxygen or peroxide catalysts in autoclaves or tube reactors. Highly branched with branches of different length, crystallinity 40 to 50%, density 0.915 to 0.935 g/cm³, average molar mass up to 600 000 g/mol.
PE-LLD (LLD=linear low density), obtainable with metal complex catalysts in the low-pressure process from the gas phase, from a solution (e.g., mineral spirit), in a suspension or with a modified high-pressure process. Slight branching with side chains which are themselves unbranched, molar masses higher than for PE-LD.
PE-HD (HD=high density), obtainable by the medium-pressure (Phillips) and low-pressure (Ziegler) processes. According to Phillips at 30 to 40 bar, 85 to 180° C., chromium oxide catalyst, molar masses about 50 000 g/mol. According to Ziegler at 1 to 50 bar, 20 to 150° C., titanium halide, titanic ester or aluminum alkyl catalysts, molar mass about 200 000 to 400 000 g/mol. Carried out in suspension, solution, gas phase or bulk. Very slight branching, crystallinity 60 to 80%, density 0.942 to 0.965 g/cm³.
PE-HD-HMW (HMW=high molecular weight), obtainable by Ziegler, Phillips or gas-phase method. High density and high molar mass.
PE-HD-UHMW (UHMW=ultra-high molecular weight) obtainable with modified Ziegler catalyst, molar mass 3 000 000 to 6 000 000 g/mol.

Particular suitability is possessed by polyethylene prepared in a gas-phase fluidized-bed process using (normally supported) catalysts, e.g., Lupolen® (Basell).

Particular suitability is also possessed by polyethylene prepared using metallocene catalysts. Polyethylene of this kind is available commercially as Luflexen® (Basell), for example.

Suitable ethylene copolymers include all commercially customary ethylene copolymers, examples being Luflexen® grades (Basell), Nordel®, and Engage® (Dow, DuPont). Examples of suitable comonomers include α-olefins having 3 to 10 carbon atoms, especially propylene, but-1-ene, hex-1-ene, and oct-1-ene, and also alkyl acrylates and methacrylates having 1 to 20 carbon atoms in the alkyl radical, especially butyl acrylate. Further suitable comonomers are dienes such as butadiene, isoprene, and octadiene, for example. Other suitable comonomers are cycloolefins, such as cyclopentene, norbornene, and dicyclopentadiene.

The ethylene copolymers are normally random copolymers or block or impact copolymers. Suitable block or impact copolymers of ethylene and comonomers are, for example, polymers for which in the first stage a homopolymer of the comonomer or a random copolymer of the comonomer is prepared, containing up to 15% by weight ethylene, for example, and then in the second stage a comonomer-ethylene copolymer with ethylene contents of 15 to 80% by weight is polymerized on. Ordinarily, sufficient of the comonomer-ethylene copolymer is polymerized on for the copolymer produced in the second stage to have a fraction of from 3 to 60% by weight in the end product.

The polymerization for preparing the ethylene-comonomer copolymers can take place by means of a Ziegler-Natta catalyst system. It is, however, also possible to use catalyst systems based on metallocene compounds or based on polymerization-active metal complexes.

Propylene Polymers:

Polypropylene should be understood below to refer both to homopolymers and to copolymers of propylene. Copolymers of propylene comprise minor amounts of monomers copolymerizable with propylene, examples being C₂-C₈ alk-1-enes such as ethylene, but-1-ene, pent-1-ene or hex-1-ene, among others. Two or more different comonomers can also be used.

Suitable polypropylenes include homopolymers of propylene or copolymers of propylene with up to 50% by weight of copolymerized other alk-1-enes having up to 8 carbon atoms. The copolymers of propylene are in this case random copolymers or block or impact copolymers. Where the copolymers of propylene are of random construction they comprise generally up to 15% by weight, preferably up to 6% by weight, of other alk-1-enes having up to 8 carbon atoms, especially ethylene, but-1-ene or a mixture of ethylene and but-1-ene.

Suitable block or impact copolymers of propylene are, for example, polymers for which in the first stage a propylene homopolymer or a random copolymer of propylene with up to 15% by weight, preferably up to 6% by weight, of other alk-1-enes having up to 8 carbon atoms is prepared and then in the second stage a propylene-ethylene copolymer having ethylene contents of from 15 to 80% by weight is polymerized on, it being possible for the propylene-ethylene copolymer further to comprise other C₄-C₈ alk-1-enes. Ordinarily, sufficient of the propylene-ethylene copolymer is polymerized on that the copolymer produced in the second stage has a fraction of from 3 to 60% by weight in the end product.

The polymerization for the preparation of polypropylene can take place by means of a Ziegler-Natta catalyst system. Use is made in particular of catalyst systems which in addition to a solid component a) containing titanium also contain cocatalysts in the form of organic aluminum compounds b) and electron donor compounds c).

It is also possible, however, to use catalyst systems based on metallocene compounds or based on polymerization-active metal complexes.

The preparation of the polypropylenes is normally carried out by polymerization in at least one reaction zone or, frequently, in two or more reaction zones connected in series (a reactor cascade), in the gas phase, in a suspension, or in a liquid phase (bulk phase). The reactors used can be the normal reactors used for polymerizing C₂-C₈ alk-1-enes. Suitable reactors include continuous stirred tanks, loop reactors, powder bed reactors or fluidized bed reactors.

The polymerization for preparing the polypropylenes used is operated under normal reaction conditions at temperatures from 40 to 120° C., in particular from 50 to 100° C., and pressures from 10 to 100 bar, in particular from 20 to 50 bar.

Suitable polypropylenes normally have a melt flow rate (MFR) in accordance with ISO 1133 of from 0.1 to 200 g/10 min., in particular from 0.2 to 100 g/10 min., at 230° C. and under a weight of 2.16 kg.

Further suitable plastics comprise at least one polyolefin. Preferred polyolefins comprise at least one copolymerized monomer selected from ethylene, propylene, but-1-ene, isobutylene, 4-methyl-1-pentene, butadiene, isoprene, and mixtures thereof. Suitability is possessed by homopolymers, copolymers of the stated olefin monomers, and copolymers of at least one of said olefins as principal monomer, with other monomers (such as vinylaromatics, for example) as comonomers.

Suitable polyolefins are low density polyethylene homopolymers (PE-LD) and polypropylene homopolymers and polypropylene copolymers. Suitable polypropylenes are, for example, biaxially oriented polypropylene (BOPP) and crystallized polypropylene.

Further suitable plastics comprise at least one polyurethane. Polyurethanes are, generally speaking, addition products of at least one diisocyanate and at least one diol component, which may also comprise higher polyfunctional isocyanates, triisocyanates for example, and higher polyfunctional polyols. Suitable isocyanates are aromatic diisocyanates such as 2,4- and 2,6-tolylene diisocyanate (TDI) and isomer mixtures thereof, tetramethylxylene diisocyanate (TMXDI), xylene diisocyanate (XDI), and diphenylmethane 4,4′-diisocyanate (MDI), and aliphatic diisocyanates, such as dicylohexylmethane 4,4′-diisocyanate (H₁₂MDI), tetramethylene diisocyanate, hexamethylene diisocyanate (HMDI), isophorone diisocyanate (IPDI), trimethylhexamethylene diisocyanate, and mixtures thereof. The preferred diisocyanates include hexamethylene diisocyanate (HMDI) and isophorone diisocyanate. Also suitable for preparing polyurethanes are triisocyanates, e.g. triphenylmethane 4,4′,4″-triisocyanate, and the cyanurates and biurets of the aforementioned diisocyanates.

Suitable diols are glycols having preferably 2 to 25 carbon atoms. These include 1,2-ethanediol, 1,2-propanediol, 1,3-propanediol, 1,4-butanediol, 1,5-pentanediol, 1,6-hexanediol, 1,10-decanediol, diethylene glycol, 2,2,4-trimethylpentane-1,5-diol, 2,2-dimethylpropane-1,3-diol, 1,4-dimethylolcyclohexane, 1,6-dimethylolcyclohexane, 2,2-bis(4-hydroxyphenyl)propane (bisphenol A), 2,2-bis(4-hydroxyphenyl)butane (bisphenol B) or 1,1-bis(4-hydroxyphenyl)-3,3,5-trimethylcyclohexane (bisphenol C).

Polyols are also useful starting materials for preparing polyurethanes. By polyols are meant trivalent alcohols (known as triols) and higher polyfunctional alcohols. They have generally 3 to 25, preferably 3 to 18, carbon atoms. They include glycerol, trimethylolethane, trimethylolpropane, erythritol, pentaerythritol, sorbitol, and the alkoxylates thereof.

Further suitable plastics may also comprise physical blends of the abovementioned polymers.

The pyridinedione derivatives of formula I are used with advantage for stabilizing thermoplastic polymers where transmittance in the uncolored state for electromagnetic radiation with a wavelength between 420 nm and 800 nm is preferably greater than 90%.

The present invention further provides for the use of at least one pyridinedione derivative of general formula I for preparing a layer which absorbs ultraviolet light. The absorbent layer is preferably transparent in the wavelength range between 420 and 800 nm.

The ultraviolet-absorbing, preferably transparent layer is based on a thermoplastic polymer. Suitable thermoplastic polymers include thermoplastics comprising at least one polymer selected from the group consisting of polyesters, polycarbonates, polyolefins, polyvinyl acetals, polystyrene, copolymers of styrene or of α-methylstyrene with dienes and/or acrylic derivatives, and also hybrid forms of the aforementioned polymers.

For example, the transparent layer may be part of an architectural or automotive glazing system or is a sheet intended for adhesive bonding to glass or plastic for the purposes of insulation or filtering; in particular it is part of a laminated window in automotive glazing. Preferably the plastic sheet comprises a polyvinyl acetal, in particular polyvinyl butyral.

The pyridinedione derivative of formula I in the laminated glass acts as a UV absorber to protect living organic and inanimate organic material, so that, for example, the driver and the inanimate organic material present in the car interior are protected against the damaging effects of ultraviolet radiation. Examples of possible damage include erythema or sunburn in the case of living organic material and yellowing, discoloration, cracking or embrittlement in the case of inanimate organic material.

Optionally the plastic further comprises at least one other light stabilizer which absorbs light radiation in the UV-A and/or UV-B region, and/or further (co)stabilizers. The light stabilizer and, if appropriate, (co)stabilizers used additionally must of course be compatible with the pyridinedione derivative of formula I. In the visible range they are preferably colorless or have only a slight inherent coloration. The light stabilizers and/or (co)stabilizers, where used, preferably have high migration fastness and temperature stability.

Suitable light stabilizers and further (co)stabilizers are selected, for example, from groups a) to s):

a) 4,4-diarylbutadienes,

b) cinnamic esters,

c) benzotriazoles,

d) hydroxybenzophenones,

e) diphenylcyanoacrylates,

f) oxamides,

g) 2-phenyl-1,3,5-triazines,

h) antioxidants,

i) nickel compounds,

j) sterically hindered amines,

k) metal deactivators,

l) phosphites and phosphonites,

m)hydroxylamines,

n) nitrones,

o) amine oxides,

p) benzofuranones and indolinones,

q) thiosynergists,

r) peroxide-destroying compounds, and

s) basic costabilizers.

Group a) of the 4,4-diarylbutadienes includes for example compounds of the formula A.

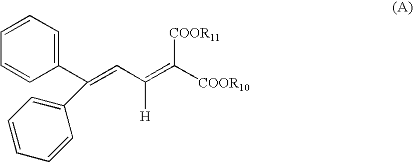

The compounds are known from EP-A-916 335. The substitutents R₁₀ and/or R₁ are preferably C₁-C₈ alkyl and C₅-C₈ cycloalkyl.

Group b) of the cinnamic esters includes for example 2-isoamyl 4-methoxycinnamate, 2-ethylhexyl 4-methoxycinnamate, methyl α-methoxycarbonylcinnamate, methyl α-cyano-α-methyl-p-methoxycinnamate, butyl α-cyano-β-methyl-p-methoxycinnamate, and methyl α-methoxycarbonyl-p-methoxycinnamate.

Group c) of the benzotriazoles includes for example 2-(2′-hydroxyphenyl)-benzotriazoles such as 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)-benzotriazole, 2-(3′,5′-bis-(α,α-dimethylbenzyl)-2′-hydroxyphenyl)benzotriazole, 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxyphenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole and 2-(3′-tert-butyl-2′-hydroxy-5′-(2-isooctyloxy-carbonylethyl)phenylbenzotriazole, 2,2′-methylenebis[4-(1,1,3,3-tetramethylbutyl)-6-benzotriazol-2-ylphenol]; the product of esterifying 2-[3′-tert-butyl-5′-(2-methoxycarbonylethyl)-2′-hydroxyphenyl]-2H-benzotriazole with polyethylene glycol 300; [R—CH₂CH₂—COO(CH₂)₃]₂ where R=3′-tert-butyl-4′-hydroxy-5′-2H-benzotriazol-2-ylphenyl and mixtures thereof.

Group d) of the hydroxybenzophenones includes for example 2-hydroxybenzophenones such as 2-hydroxy-4-methoxybenzophenone, 2,2′-dihydroxy-4-methoxybenzophenone, 2,4-dihydroxybenzophenone, 2,2′,4,4′-tetra-hydroxybenzophenone, 2,2′-dihydroxy-4,4′-dimethoxybenzophenone, 2,2′-dihydroxy-4,4′-dimethoxybenzophenone, 2-hydroxy-4-(2-ethylhexyloxy)benzophenone, 2-hydroxy-4-(n-octyloxy)benzophenone, 2-hydroxy-4-methoxy-4′-methylbenzophenone, 2-hydroxy-3-carboxybenzophenone, 2-hydroxy-4-methoxybenzophenone-5-sulfonic acid and its sodium salt, and 2,2′-dihydroxy-4,4′-dimethoxybenzophenone-5,5′-bissulfonic acid and its sodium salt.

Group e) of the diphenylcyanoacrylates includes for example ethyl 2-cyano-3,3-diphenylacrylate, obtainable commercially for example under the name Uvinul® 3035 from BASF AG, Ludwigshafen, 2-ethylhexyl 2-cyano-3,3-diphenylacrylate, obtainable commercially for example as Uvinul® 3039 from BASF AG, Ludwigshafen, and 1,3-bis[(2′-cyano-3′,3′-diphenylacryloyl)oxy]-2,2-bis{[2′-cyano-3′,3′-diphenyl-acryloyl)oxy]methyl}propane, obtainable commercially for example under the name Uvinul® 3030 from BASF AG, Ludwigshafen.

Group f) of the oxamides includes for example 4, 4′-dioctyloxyoxanilide, 2,2′-di-ethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, and also mixtures of ortho-, para-methoxy-disubstituted oxanilides and mixtures of ortho- and para-ethoxy-disubstituted oxanilides.

Group g) of the 2-phenyl-1,3,5-triazines includes for example 2-(2-hydroxyphenyl)-1,3,5-triazines such as 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropoxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis-(2,4-dimethylphenyl)-1,3,5-triazine, 2-(2-hydroxy-4-hexyloxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, and 2-(2-hydroxyphenyl)-4-(4-methoxyphenyl)-6-phenyl-1,3,5-triazine.

Group h) of the antioxidants comprises, for example:

Alkylated monophenols such as, for example, 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, unbranched or sidechain-branched nonylphenols such as, for example, 2,6-dinonyl-4-methylphenol, 2,4-dimethyl-6-(1-methylundec-1-yl)phenol, 2,4-dimethyl-6-(1-methylheptadec-1-yl)phenol, 2,4-dimethyl-6-(1-methyltridec-1-yl)phenol, and mixtures thereof.

Alkylthiomethylphenols such as, for example, 2,4-dioctylthiomethyl-6-tert-butylphenol, 2,4-dioctylthiomethyl-6-methylphenol, 2,4-dioctylthiomethyl-6-ethylphenol and 2,6-didodecylthiomethyl-4-nonylphenol.

Hydroquinones and alkylated hydroquinones such as, for example, 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, and bis-(3,5-di-tert-butyl-4-hydroxyphenyl) adipate.

Tocopherols, such as, for example, α-tocopherol, α-tocopherol, γ-tocopherol, δ-tocopherol, and mixtures thereof (vitamin E).

Hydroxylated thiodiphenyl ethers such as, for example, 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), and 4,4′-bis(2,6-dimethyl-4-hydroxyphenyl)disulfide.

Alkylidenebisphenols such as, for example, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethylene glycol bis[3,3-bis(3-tert-butyl-4-hydroxyphenyl)butyrate], bis(3-tert-butyl-4-hydroxy-5-methyl-phenyl)dicyclopentadiene, bis[2-(3′-tert-butyl-2-hydroxy-5-methylbenzyl)-6-tert-butyl-4-methylphenyl]terephthalate, 1,1-bis(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra-(5-tert-butyl-4-hydroxy-2-methylphenyl)pentane.

Benzyl compounds such as, for example, 3,5,3′,5′-tetra-tert-butyl-4,4′-dihydroxydibenzyl ether, octadecyl 4-hydroxy-3,5-dimethylbenzylmercaptoacetate, tridecyl 4-hydroxy-3,5-di-tert-butylbenzylmercaptoacetate, tris(3,5-di-tert-butyl-4-hydroxybenzyl)amine, 1,3,5-tri(3,5-di-tert-butyl-4-hydroxybenzyl)-2,4,6-trimethylbenzene, di(3,5-di-tert-butyl-4-hydroxybenzyl)sulfide, isooctyl 3,5-di-tert-butyl-4-hydroxybenzylmercaptoacetate, bis(4-tert-butyl-3-hydroxy-2,6-dimethylbenzyl)dithiol terephthalate, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 3,5-di-tert-butyl-4-hydroxybenzyl-phosphoric acid dioctadecyl ester, and 3,5-di-tert-butyl-4-hydroxybenzyl-phosphoric acid monoethyl ester, calcium salt.

Hydroxybenzylated malonates such as, for example, dioctadecyl 2,2-bis(3,5-di-tert butyl-2-hydroxybenzyl)malonate, dioctadecyl-2-(3-tert-butyl-4-hydroxy-5-methylbenzyl)malonate, didodecyl mercaptoethyl-2,2-bis(3,5-di-tert-butyl-4-hydroxybenzyl)malonate, and bis[4-(1,1,3,3-tetramethylbutyl)phenyl] 2,2-bis(3,5-di-tert-butyl-4-hydroxybenzyl)malonate.

Hydroxybenzyl aromatics such as, for example, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, and 2,4,6-tris(3,5-di-tert-butyl-4-hydroxybenzyl)phenol.

Triazine compounds such as, for example, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)hexahydro-1,3,5-triazine, and 1,3,5-tris(3,5-dicyclohexyl-4-hydroxybenzyl)isocyanurate.

Benzylphosphonates such as, for example, dimethyl 2,5-di-tert-butyl-4-hydroxybenzylphosphonate, diethyl 3,5-di-tert-butyl-4-hydroxybenzylphosphonate (diethyl (3,5-bis(1,1-dimethylethyl)-4-hydroxyphenyl)methylphosphonate), dioctadecyl 3,5-di-tert-butyl-4-hydroxybenzylphosphonate, dioctadecyl 5-tert-butyl-4-hydroxy-3-methylbenzylphosphonate, and the calcium salt of monoethyl 3,5-di-tert-butyl-4-hydroxybenzylphosphonate.

Acylaminophenols such as, for example, 4-hydroxylauranilide, 4-hydroxystearanilide, 2,4-bisoctylmercapto-6-(3,5-tert-butyl-4-hydroxyanilino)-s-triazine, and octyl N-(3,5-di-tert-butyl-4-hydroxyphenyl)carbamate.

Esters of β-(3,5-di-tert-butyl-4-hydroxyphenyl)propionic acid with monohydric or polyhydric alcohols, such as with methanol, ethanol, n-octanol, isooctanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane, and 4-hydroxymethyl-1-phospha-2,6,7-trioxabicyclo[2.2.2]octane.

Esters of α-(5-tert-butyl-4-hydroxy-3-methylphenyl)propionic acid with monohydric or polyhydric alcohols, such as with methanol, ethanol, n-octanol, isooctanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane, and 4-hydroxymethyl-1-phospha-2,6,7-trioxabicyclo[2.2.2]octane.

Esters of β-(3,5-dicyclohexyl-4-hydroxyphenyl)propionic acid with monohydric or polyhydric alcohols, such as with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane, and 4-hydroxymethyl-1-phospha-2,6,7-trioxabicyclo[2.2.2]octane.

Esters of 3,5-di-tert-butyl-4-hydroxyphenylactic acid with monohydric or polyhydric alcohols, such as with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane, and 4-hydroxymethyl-1-phospha-2,6,7-trioxabicyclo[2.2.2]octane.

Amides of β-(3,5-di-tert-butyl-4-hydroxyphenyl)propionic acid, such as N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (e.g. Naugard®XL-1 from Uniroyal).

Ascorbic Acid (Vitamin C)

Amine antioxidants, such as, for example, N,N′-diisopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxy-diphenylamine, N-phenyl-1-naphthylamine, N-(4-tert-octylphenyl)-1-naphthylamine, N-phenyl-2-naphthylamine, octylated diphenylamine, for example, p,p′-di-tert-octyidiphenylamine, 4-n-butylaminophenol, 4-butyrylaminophenol, 4-nonanoyl-aminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N, N′,N′-tetramethyl-4,4′-diaminodiphenylmethane, 1,2-bis[(2-methylphenyl)amino]ethane, 1,2-bis(phenylamino)propane, o-tolyl biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazines, N-allylphenothiazine, N,N,N′,N′-tetraphenyl-1,4-diaminobut-2-ene, N,N-bis(2,2,6,6-tetramethylpiperidin-4-yl)hexamethylenediamine, bis(2,2,6,6-tetramethylpiperidin-4-yl) sebacate, 2,2,6,6-tetramethylpiperidin-4-one, 2,2,6,6-tetramethylpiperidin-4-ol, the dimethyl succinate polymer with 4-hydroxy-2,2,6,6-tetramethyl-1-piperidineethanol [CAS number 65447-77-0], (for example, Tinuvin® 622 from Ciba Specialty Chemicals, Inc.), polymer of 2,2,4,4-tetramethyl-7-oxa-3,20-diazadispiro[5.1.11.2]heneicosan-21-one and epichlorohydrin [CAS No.: 202483-55-4], for example (Hostavin®30 from Ciba Specialty Chemicals, Inc.).

Group i) of the nickel compounds includes for example nickel complexes of 2,2′-thiobis[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyl dithiocarbamate, nickel salts of 4-hydroxy-3,5-di-tert-butylbenzylphosphonic acid monoalkyl esters such as of the methyl or ethyl esters, for example, nickel complexes of ketoximes such as, for example, of 2-hydroxy-4-methylphenyl undecyl ketoxime, and the nickel complex of 1-phenyl-4-lauroyl-5-hydroxypyrazole, with or without additional ligands.

Group j) of the sterically hindered amines includes for example 4-hydroxy-2,2,6,6-tetramethylpiperidine, 1-allyl-4-hydroxy-2,2,6,6-tetramethylpiperidine, 1-benzyl-4-hydroxy-2,2,6,6-tetramethylpiperidine, bis(2,2,6,6-tetramethyl-4-piperidyl)sebacate, bis(2,2,6,6-tetramethyl-4-piperidyl)succinate, bis(1,2,2,6,6-pentamethyl-4-piperidyl) sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate (n-butyl-3,5-di-tert-butyl-4-hydroxybenzylmalonic acid bis(1,2,2,6,6-pentamethylpiperidyl)ester), condensation product of 1-(2-hydroxyethyl)-2,2,6,6-tetramethyl-4-hydroxypiperidine and succinic acid, linear or cyclic condensation products of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl) 1,2,3,4-butanetetracarboxylate, 1,1′-(1,2-ethanediyl)bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)2-n-butyl-2-(2-hydroxy-3,5-di-tert-butylbenzyl)malonate, 3-n-octyl-7,7,9,9-tetramethyl-1,3,8-triazaspiro[4,5]decane-2,4-dione, bis(1-octyloxy-2,2,6,6-tetramethylpiperidyl)sebacate, bis(1-octyloxy-2,2,6,6-tetramethylpiperidyl)succinate, linear or cyclic condensation products of N,N′-bis(2,2,6,6-tetramethyl-4-piperidyl)hexamethylenediamine and 4-morpholino-2,6-dichloro-1,3,5-triazine, condensation product of N,N′-bis(2,2,6,6-tetramethyl-4-piperidyl)hexamethylenediamine and formic esters (CAS No. 124172-53-8, e.g., Uvinul® 4050H from BASF AG, Ludwigshafen), condensation product of 2-chloro-4,6-bis(4-n-butylamino-2,2,6,6-tetramethylpiperidyl)-1,3,5-triazine and 1,2-bis(3-aminopropylamino)ethane, condensation product of 2-chloro-4,6-di-(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4,5]decane-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)pyrrolidine-2,5-dione, mixture of 4-hexadecyloxy- and 4-stearyloxy-2,2,6,6-tetramethylpiperidine, condensation product of N,N′-bis(2,2,6,6-tetramethyl-4-piperidyl)hexamethylenediamine and 4-cyclohexylamino-2,6-dichloro-1,3,5-triazine, condensation product of 1,2-bis(3-aminopropylamino)ethane and 2,4,6-trichloro-1,3,5-triazine and also 4-butylamino-2,2,6,6-tetramethylpiperidine (CAS Reg. No. [136504-96-6]); N-(2,2,6,6-tetramethyl-4-piperidyl)-n-dodecylsuccinimide, N-(1,2,2,6,6-pentamethyl-4-piperidyl)-n-dodecylsuccinimide, 2-undecyl-7,7,9,9-tetramethyl-1-oxa-3,8-diaza-4-oxospiro[4,5]decane, reaction product of 7,7,9,9-tetramethyl-2-cycloundecyl-1-oxa-3,8-diaza-4-oxospiro[4,5]decane and epichlorohydrin, 1,1-bis(1,2,2,6,6-pentamethyl-4-piperidyloxycarbonyl)-2-(4-methoxyphenyl)ethene, N,N′-bisformyl-N,N′-bis(2,2,6,6-tetramethyl-4-piperidyl)hexamethylenediamine, diester of 4-methoxymethylenemalonic acid with 1,2,2,6,6-pentamethyl-4-hydroxypiperidine, poly[methylpropyl-3-oxo-4-(2,2,6,6-tetramethyl-4-piperidyl)]siloxane, reaction product of maleic anhydride-α-olefin copolymer and 2,2,6,6-tetramethyl-4-aminopiperidine or 1,2,2,6,6-pentamethyl-4-aminopiperidine, copolymers of (partially) N-piperidin-4-yl-substituted maleimide and a mixture of α-olefins such as Uvinul® 5050H (BASF AG), 1-(2-hydroxy-2-methylpropoxy)-4-octadecanoyloxy-2,2,6,6-tetramethylpiperidine, 1-(2-hydroxy-2-methylpropoxy)-4-hexadecanoyloxy-2,2,6,6-tetramethylpiperidine, the reaction product of 1-oxyl-4-hydroxy-2,2,6,6-tetramethylpiperidine and a carbon radical of t-amyl alcohol, 1-(2-hydroxy-2-methylpropoxy)-4-hydroxy-2,2,6,6-tetramethylpiperidine, 1-(2-hydroxy-2-methylpropoxy)-4-oxo-2,2,6,6-tetramethylpiperidine, bis(1-(2-hydroxy-2-methylpropoxy)-2,2,6,6-tetramethylpiperidin-4-yl)sebacate, bis(1-(2-hydroxy-2-methylpropoxy)-2,2,6,6-tetramethylpiperidin-4-yl)adipate, bis(1-(2-hydroxy-2-methylpropoxy)-2,2,6,6-tetramethylpiperidin-4-yl)succinate, bis(1-(2-hydroxy-2-methylpropoxy)-2,2,6,6-tetramethylpiperidin-4-yl)glutarate, 2,4-bis{N[1-(2-hydroxy-2-methylpropoxy)-2,2,6,6-tetramethylpiperidin-4-yl]-N-butylamino}-6-(2-hydroxyethylamino)-s-triazine, N,N′-bisformyl-N,N′-bis(1,2,2,6,6-pentamethyl-4-piperidyl)hexamethylenediamine, hexahydro-2,6-bis(2,2,6,6-tetramethyl-4-piperidyl)-1H,4H,5H,8H-2,3a,4a,6,7a,8a-hexaazacyclopenta[def]fluorene-4,8-dione (e.g. Uvinul® 4049 from BASF AG, Ludwigshafen), poly[[6-[(1,1,3,3-tetramethylbutyl)amino]-1,3,5-triazine-2,4-diyl][(2,2,6,6-tetramethyl-4-piperidinyl)imino]-1,6-hexanediyl[(2,2,6,6-tetramethyl-4-piperidinyl)imino]]) [CAS No. 71878-19-8], 1,3,5-triazine-2,4,6-triamine, N,N′″-[1,2-ethanediylbis[[4,6-bis[butyl(1,2,2,6,6-pentamethyl-4-piperidinyl)amino]-1,3,5-triazin-2-yl]imino]-3,1-propanediyl]]bis[N′,N″-dibutyl-N′,N″-bis(1,2,2,6,6-pentamethyl-4-piperidinyl) (CAS No. 10699043-6) (e.g., Chimassorb 119 from Ciba Specialty Chemicals, Inc.).

Group k) of the metal deactivators includes for example N,N′-diphenyloxalamide, N-salicylal-N′-salicyloylhydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoylbisphenyl hydrazide, N,N′-diacetyladipic dihydrazide, N,N′-bis(salicyloyl)oxalic dihydrazide, and N,N′-bis(salicyloyl)thiopropionyl dihydrazide.

Group l) of the phosphites and phosphonites includes for example triphenyl phosphite, diphenyl alkyl phosphites, phenyl dialkyl phosphites, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl) pentaerythritol diphosphite, diisodecyloxy pentaerythritol diphosphite, bis(2,4-di-tert-butyl-6-methylphenyl)pentaerythritol diphosphite, bis(2,4,6-tris(tert-butylphenyl) pentaerythritol diphosphite, tristearyl sorbitol triphosphite, tetrakis(2,4-di-tert-butylphenyl) 4,4′-biphenylenediphosphonite, 6-isooctyloxy-2,4,8,10-tetra-tert-butyl-dibenzo[d,f][1,3,2]dioxaphosphepine, 6-fluoro-2,4,8,10-tetra-tert-butyl-1,2-methyl-dibenzo[d,g][1,3,2]dioxaphosphocine, bis(2,4-di-tert-butyl-6-methylphenyl)methyl phosphite, bis(2,4-di-tert-butyl-6-methylphenyl)ethyl phosphite, 2,2′,2″-nitrilo[triethyl tris(3,3′,5,5′-tetra-tert-butyl-1,1′-biphenyl-2,2′-diyl)phosphite], and 2-ethylhexyl(3,3′,5,5′-tetra-tert-butyl-1,1′-biphenyl-2,2′-diyl)phosphite.

Group m) of the hydroxylamines includes for example N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecyl-hydroxylamine, N-hexadecyl-N-octadecylhydroxylamine, N-heptadecyl-N-octadecylhydroxylamine, N-methyl-N-octadecylhydroxylamine, and N,N-dialkylhydroxylamine from hydrogenated tallow fatty amines.

Group n) of the nitrones includes for example N-benzyl α-phenyl nitrone, N-ethyl α-methyl nitrone, N-octyl α-heptyl nitrone, N-lauryl α-undecyl nitrone, N-tetradecyl α-tridecyl nitrone, N-hexadecyl α-pentadecyl nitrone, N-octadecyl α-heptadecyl nitrone, N-hexadecyl α-heptadecyl nitrone, N-octadecyl α-pentadecyl nitrone, N-heptadecyl α-heptadecyl nitrone, N-octadecyl α-hexadecyl nitrone, N-methyl α-heptadecyl nitrone, and nitrones derived from N,N-dialkylhydroxylamines prepared from hydrogenated talc fatty amines.

Group o) of the amine oxides includes for example amine oxide derivatives as described in U.S. Pat. Nos. 5,844,029 and 5,880,191, didecylmethylamine oxide, tridecylamine oxide, tridodecylamine oxide and trihexadecylamine oxide.

Group p) of the benzofuranones and indolinones includes for example those described in U.S. Pat. Nos. 4,325,863; 4,338,244; 5,175,312; 5,216,052; 5,252,643; in DE-A4316611; in DE-A4316622; in DE-A4316876; in EP-A-0589839 or EP-A-0591102, or 3-[4-(2-acetoxyethoxy)phenyl]-5,7-di-tert-butylbenzofuran-2-one, 5,7-di-tert-butyl-3-[4-(2-stearoyloxyethoxy)phenyl]benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, Irganoxs HP-136 from Ciba Specialty Chemicals, and 3-(2,3-dimethylphenyl)-5,7-di-tert-butyl-benzofuran-2-one.

Group q) of the thiosynergists includes for example dilauryl thiodipropionate or distearyl thiodipropionate.

Group r) of the peroxide-destroying compounds includes for example esters of β-thiodipropionic acid, for example, the lauryl, stearyl, myristyl or tridecyl ester, mercaptobenzimidazole or the zinc salt of 2-mercaptobenzimidazole, zinc dibutyldithiocarbamate, dioctadecyl disulfide, and pentaerythritol tetrakis(β-dodecylmercapto)propionate.

Group s) of the basic costabilizers includes for example melamine, polyvinylpyrrolidone, dicyandiamide, triallylcyanurate, urea derivatives, hydrazine derivatives, amines, polyamides, polyurethanes, alkali metal and alkaline earth metal salts of higher fatty acids, for example, calcium stearate, zinc stearate, magnesium behenate, magnesium stearate, sodium ricinoleate, and potassium palmitate, antimony pyrocatecholate or zinc pyrocatecholate.

It is preferred to use at least one compound I together with at least one further light stabilizer having at least one absorption maximum in the wavelength range from 280 to 400 nm. The further light stabilizer is preferably selected from compounds of groups b), c), d), e) and g).

The light stabilizer in question in particular has at least one absorption maximum in the wavelength range from 280 to 320 nm. Accordingly, the light stabilizer employed additionally has at least one absorption maximum in the UVB range. Absorption maxima for the purposes of the present invention are the bands associated with the corresponding local or absolute maxima in the UV spectrum of the respective compounds, as measured in common organic solvents such as dichloromethane, acetonitrile or methanol at room temperature. The extinction of the UVB absorbers at the maximum, which is measured in solution, normally in dichloromethane, at a concentration of 1% by weight and a path length of 1 cm, is at least 100, in particular at least 200.

Examples of light stabilizers used additionally in particular are the aforementioned diphenylcyanoacrylates of group e).

The plastic may further comprise other additives and auxiliaries. Suitable additives from the group t) are the customary additives, such as pigments, dyes, nucleating agents, fillers, reinforcing agents, antifogging agents, biocides, and antistats, for example.

Suitable pigments are inorganic pigments, examples being titanium dioxide in its three modifications—rutile, anatase or brookite; ultramarine blue, iron oxides, bismuth vanadates or carbon black, and also the class of the organic pigments, examples being compounds from the class of the phthalocyanines, perylenes, azo compounds, isoindolines, quinophthalones, diketopyrrolopyrroles, quinacridones, dioxazines, and indanthrones.

By dyes are meant all colorants which dissolve completely in the plastic used or are present in a molecularly disperse distribution and can therefore be used for the high-transparency, nonscattering coloring of polymers. Likewise regarded as dyes are organic compounds which exhibit a fluorescence in the visible part of the electromagnetic spectrum, such as fluorescent dyes.

Suitable nucleating agents comprise for example inorganic substances, examples being talc, metal oxides such as titanium dioxide or magnesium oxide, phosphates, carbonates or sulfates, preferably of alkaline earth metals; organic compounds such as monocarboxylic or polycarboxylic acids and also their salts, such as 4-tert-butylbenzoic acid, adipic acid, diphenylactic acid, sodium succinate or sodium benzoate; and polymeric compounds, such as ionic copolymers (“ionomers”), for example.

Suitable fillers and reinforcing agents comprise for example calcium carbonate, silicates, talc, mica, kaolin, barium sulfate, metal oxides and metal hydroxides, carbon black, graphite, wood flour and flours or fibers of other natural products, and synthetic fibers. Further suitable examples of fibrous or pulverulent fillers include carbon or glass fibers in the form of glass fabrics, glass mats or filament glass rovings, chopped glass, glass beads, and wollastonite. Glass fibers can be incorporated either in the form of short glass fibers or in the form of continuous fibers (rovings).

Examples of suitable antistats include amine derivatives such as N,N-bis(hydroxy-alkyl)alkylamines or -alkylenamines, polyethylene glycol esters and ethers, ethoxylated carboxylic esters and carboxamides, and glyceryl mono- and distearates, and also mixtures thereof.

Normally the plastic is admixed with at least one pyridinedione derivative of formula I in an amount of from 0.01 to 10% by weight, preferably from 0.01 to 5% by weight, and more preferably from 0.01 to 1.0% by weight, based on the total weight of the plastic. By the total weight of the plastic is meant the weight of the plastic additized with the pyridinedione derivative of formula I and, if appropriate, with further (co)stabilizers (plastic+sum of all (co)stabilizers+sum of all other additives). The light protection achieved is dependent on the path length in the plastic. This is illustrated by the Lambert-Beer law E=ε·c·d (ε: molar extinction (absorbance) coefficient, c: concentration, d: path length). In thin layers of plastic, therefore, it is usual to use a higher proportion of UV absorber than in a thick layer of plastic.

The compounds from groups a) to s) are used in customary amounts known to the skilled worker. Generally they are employed at a concentration of from 0.0001 to 10% by weight, preferably from 0.01 to 1% by weight, based on the total weight of the plastic. In the case of the group p) benzofuranones it is usual to use even lower concentrations of preferably 0.001 to 0.1% by weight.

The additives of group t) are used in the customary amounts. They are normally used in an amount of from 0 to 60% by weight, based on the total weight of the plastic.

The pyridinedione derivative of formula I used in accordance with the invention can also be added in the form of a premix (masterbatch or compound) comprising at least one pyridinedione derivative of formula I in a concentration of from 1 to 20% by weight to the materials that are to be stabilized, usually a plastic. The premix may further comprise the aforementioned compounds of groups a) to s) and other additives of group t).

The present invention additionally provides compositions comprising at least one pyridinedione derivative of formula I as defined above in an amount providing protection from the damaging effects of light, and at least one organic material. The organic material is preferably a polymer selected from the group consisting of polyesters, polycarbonate polymers, polyolefins, polyvinyl acetals, polystyrene, copolymers of styrene or of α-methylstyrene with dienes and/or acrylic derivatives, and physical blends of the aforementioned polymers.

The text below relating to the compositions of the invention concerning suitable and preferred embodiments applies equally to the corresponding use of such a pyridinedione derivative of formula I in a thermoplastic polymer of this kind.

One advantageous composition comprises for example:
at least one pyridinedione derivative of formula I as defined above;
at least one polyvinyl butyral (PVB);
at least one oligoalkylene glycol alkylcarboxylic diester as plasticizer;
at least one aliphatic carboxylic salt to control the adhesion;
if appropriate, at least one further UV absorber selected from the group consisting of benzotriazoles, 2-phenyl-1,3,5-triazines, hydroxybenzophenones, diphenylcyanoacrylates, and mixtures thereof; and
if appropriate, at least one further component selected from the group consisting of fillers, dyes, pigments, and additional additives.

Preference is given to employing at least one pyridinedione derivative of formula I in PVB sheets in laminated glass, for automotive glazing systems for example.

With regard to the preparation of polyvinyl butyral, the text above is incorporated in its entirety by reference. The polyvinyl butyrals used in the polymer composition generally have an average molecular mass of more than 70 000, preferably from about 100 000 to 250 000. The polyvinyl butyral normally has a residual hydroxyl group content of less than 19.5%, preferably from about 17 to 19% by weight, calculated as polyvinyl alcohol, and a residual ester group content of from 0 to 10%, preferably from 0 to 3%, calculated as polyvinyl ester. An advantageous polyvinyl butyral is that obtainable under the name Butvar® from Solutia, Inc. of St. Louis, Mo. The polyvinyl butyral molding compound is normally used in the form of a sheet with a thickness of from 0.13 to 1.5 mm. The polyvinyl butyral can be shaped to the desired thickness on a sheet extrusion line, for example.

Suitable oligoalkylene glycol carboxylic diesters comprise the esters of aliphatic, unbranched or branched C₂-C₁₀ monocarboxylic acids, preferably C₆-C₈ monocarboxylic acids, with tri-C₂-C₃ alkylene glycols or tetra-C₂-C₃ alkylene glycols. Suitable plasticizers are, for example, triethylene glycol di(2-ethylbutyrate), triethylene glycol di(2-ethylhexanoate), triethylene glycol diheptanoate or tetraethylene glycol diheptanoate. The fraction of plasticizer is generally from 20 to 80% by weight, preferably from 25 to 45% by weight, based on the total weight of the polymer composition.

Suitable aliphatic carboxylic salts to control adhesion are, for example, the polyvalent metal salts of branched or unbranched C₄-C₂₂ monocarboxylic acids. Suitable metals include, for example, zinc, aluminum, lead or alkaline earth metals such as magnesium or calcium. A suitable example of an aliphatic carboxylic salt to control adhesion is, for example, the magnesium salt of 2-ethylbutyric acid. The salts lower the tack and viscosity of the polyvinyl butyral. The fraction of aliphatic carboxylic salt is generally from 0.0001 to 0.5% by weight, preferably from 0.0001 to 0.1% by weight, based on the total weight of the polymer composition.

The polyvinyl butyral polymer composition may further comprise at least one additional UV absorber, preferably selected from the group consisting of benzotriazoles, 2-phenyl-1,3,5-triazines, hydroxybenzophenones, diphenylcyanoacrylates, and mixtures thereof.

Examples of suitable benzotriazoles are 2-(2′-hydroxyphenyl)benzotriazoles, preferably those mentioned above. Particular preference is given to the following:
2-(3′-tert-butyl-2′-hydroxy-5′-methylphenyl)-5-chlorobenzotriazole [CAS No. 3896-11-5), available commerically for example as Tinuvin® 326 from Ciba Specialty Chemicals, Inc.;
2,4-di-tert-butyl-6-(5-chlorobenzotriazol-2-yl)phenol [CAS No. 3864-99-1], available commercially for example as Tinuvin® 327 from Ciba Specialty Chemicals, Inc.;
2-(2H-benzotriazol-2-yl)-4,6-di-tert-pentylphenol [CAS No. 25973-55-1], available commercially for example as Tinuvin® 328 from Ciba Specialty Chemicals, Inc.; and
2-benzotriazol-2-yl-4-methylphenol [CAS No. 2440-224], available commercially for example as Tinuvin® P from Ciba Specialty Chemicals, Inc.

Examples of suitable 2-phenyl-1,3,5-triazines are 2-(2′-hydroxyphenyl)-1,3,5-triazines, preferably those mentioned above. Particular preference is given to the following:
2-(4,6-diphenyl-1,3,5-triazin-2-yl)-5-hexyloxyphenol [CAS 147315-50-2], available commercially for example as Tinuvin® 1577 from Ciba Specialty Chemicals, Inc.; and
2,4-bis(2,4-dimethylphenyl)-6-(2-hydroxy-4-octyloxyphenyl)-1,3,5-triazine [CAS No. 2725-22-6], available commercially for example as Cyasorb® UV 1164 from Cytec.

Examples of suitable hydroxybenzophenones are 2-hydroxybenzophenones, preferably those mentioned above. Particular preference is given to the following:
2-hydroxy-4-n-octoxybenzophenone [CAS No. 1843-05-6], available commercially for example as Chimassorb® 81 from Ciba Specialty Chemicals, Inc.

Examples of suitable diphenylcyanoacrylates are those mentioned above. Particular preference is given to the following:
1,3-bis[(2′-cyano-3′,3′-diphenylacryloyl)oxy]-2,2-bis {[2′-cyano-3′,3′-diphenyl-acryloyl)oxy]methyl}propane [CAS No. 178671-58-4], available commercially for example under the name Uvinul® 3030 from BASF AG, Ludwigshafen;
ethyl 2-cyano-3,3-diphenylacrylate [CAS No. 5232-99-5], available commercially for example under the name Uvinul® 3035 from BASF AG, Ludwigshafen; and
2-ethylhexyl 2-cyano-3,3-diphenylcyanoacrylate [CAS 6197-30-4], available commercially for example under the name Uvinul® 3039 from BASF AG, Ludwigshafen.

Generally speaking, the fraction of further UV absorber, dependent on the thickness of the sheet used, is from 0.05 to 2% by weight, preferably from 0.1 to 1% by weight, based on the total weight of the polymer composition. In the case of thin polymer layers the fraction of UV absorber used is generally higher than in the case of thick polymer layers.

The polyvinyl butyral polymer composition may further comprise at least one additional component selected from the group consisting of fillers, dyes, pigments, and further additives. As regards suitable fillers, dyes, and pigments, the text above is incorporated in its entirety by reference.

Another advantageous composition comprises:
at least one pyridinedione derivative of formula I as defined above;
at least one polycarbonate polymer selected from the group consisting of polycarbonates, polycarbonate copolymers, and physical blends of polycarbonates with acrylic-butadiene-styrene copolymers, acrylonitrile-styrene-acrylate copolymers, polymethyl methacrylates, polybutyl acrylates, polybutyl methacrylates, poly(butylene terephthalate)s, and polyethylene terephthalates;
at least one stabilizer selected from the group consisting of phosphites, phosphonites, and mixtures thereof;
if appropriate, at least one further UV absorber selected from the group consisting of benzotriazoles, 2-phenyl-1,3,5-triazines, diphenylcyanoacrylates, and mixtures thereof;
if appropriate, at least one 2,6-dialkylated phenol antioxidant, and
if appropriate, at least one further component selected from the group consisting of fillers, dyes, pigments, and other additives.

Advantageously the pyridinedione derivatives of formula I also find use in polycarbonate polymer compositions.

For the purposes of the present specification the term “polycarbonate copolymers” comprises polycarbonates obtainable by condensing phosgene or carbonic esters with at least two different dihydroxy compounds: different bisphenols, for example. A fraction of halogenated bisphenols, tetrabromobisphenol for example, raises the flame retardancy; a fraction of bisphenol S (dihydroxydiphenyl sulfide) raises the notched impact strength. The polycarbonate copolymers include for example polycarbonate copolymers based on bisphenol A and bisphenol C, or polycarbonate copolymers based on bisphenol A and bisphenol TMC (trimethylcyclohexane). For the purposes of the present invention the term “polycarbonate copolymers” also comprises polyester carbonates, which are obtainable for example by reacting bisphenols with phosgene and aromatic dicarbonyl dichlorides, and block copolymers comprising polycarbonate blocks and polyalkylene oxide blocks.

The polycarbonate polymer composition comprises at least one stabilizer selected from the group consisting of phosphites and phosphonites. As regards suitable phosphites and phosphonites, the remarks above are incorporated in their entirety by reference. Preferred phosphites and phosphonites are tris(2,4-di-tert-butylphenyl)phosphite [CAS No. 31570-04-4], which is available commercially for example as Irgafos® 168 from Ciba Specialty Chemicals, Inc., tetrakis(2,4-di-tert-butylphenyl)-4,4′-diyl bisphosphonite [CAS No. 119345-01-6], obtainable commercially for example as Irgafos® P-EPQ from Ciba Specialty Chemicals, Inc., and mixtures thereof. The fraction of phosphite and/or phosphonite is generally up to 2000 ppm, preferably from 500 to 1500 ppm, based on the total weight of the polymer composition.

The polycarbonate polymer composition may further comprise at least one other UV absorber. Suitable other UV absorbers are those mentioned above. The other UV absorbers are preferably selected from the group consisting of benzotriazoles, 2-phenyl-1,3,5-triazines, diphenylcyanoacrylates, and mixtures thereof.

Examples of suitable benzotriazoles are 2-(2′-hydroxyphenyl)benzotriazoles, preferably those mentioned above. Particular preference is given to the following:
2-(2H-benzotriazol-2-yl)-4,6-bis(1-methyl-1-phenylethyl)phenol [CAS No. 70321-86-7], available commercially for example as Tinuvin® 23-4 from Ciba Specialty Chemicals, Inc.;
2,4-di-tert-butyl-6-(5-chlorobenzotriazol-2-yl)phenol [CAS No. 3864-99-1], available commercially for example as Tinuvin® 327 from Ciba Specialty Chemicals, Inc.;
2-(2H-benzotriazol-2-yl)-4-(1,1,3,3-tetramethylbutyl)phenol [CAS No. 3147-75-9], available commercially for example as Tinuvin® 329 from Ciba Specialty Chemicals, Inc.;
2-(2H-benzotriazol-2-yl)-4-(tert-butyl)-6-(sec-butyl)phenol [CAS No. 36437-37-3], available commercially for example as Tinuvin® 350 from Ciba Specialty Chemicals, Inc.;
2,2′-methylenebis-(6-(2H-benzotriazol-2-yl)-4-1,1,3,3-tetramethylbutyl)phenol) [CAS No. 10359745-1], available commercially for example as Tinuvin® 360 from Ciba Specialty Chemicals, Inc.; and
transesterification products of methyl 3-(3-(2H-benzotriazol-2-yl)-5-tert-butyl-4-hydroxyphenyl)propionate with polyethylene glycol, obtainable for example as Tinuvin® 213 from Ciba Specialty Chemicals, Inc., (comprising 52% of the compound R—COO—[(CH₂)₂—O]ₙ—H (molar mass: 637 g/mol) [CAS No. 10481048-2], 35% of the compound of the formula R—COO—[(CH₂)₂—O]ₙ—CO—R (molar mass: 975 g/mol) [CAS No. 104810-47-1] with 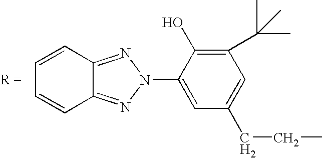
   and 13% of the compound HO—[(CH₂)₂—O]ₙ—H [CAS No. 25322-68-3].

Examples of 2-phenyl-1,3,5-triazines are 2-(2′-hydroxyphenyl)-1,3,5-triazines, preferably those mentioned above. Particular preference is given to the following:
2-(4,6-diphenyl-1,3,5-triazin-2-yl)-5-hexyloxyphenol [CAS No. 147315-50-2], available commercially for example as Tinuvin® 1577 from Ciba Specialty Chemicals, Inc.

Examples of suitable diphenylcyanoacrylates are those mentioned above. Preference is given to the following:
1,3-bis[(2′-cyano-3′,3′-diphenylacryloyl)oxy]-2,2-bis{[2′-cyano-3′,3′-diphenyl-acryloyl)oxy]methyl}propane [CAS No. 178671-58-4], available commercially for example under the name Uvinul® 3030 from BASF AG, Ludwigshafen; and
ethyl-2-cyano-3,3-diphenylacrylate [CAS No. 5232-99-5], available commercially for example under the name Uvinul® 3035 from BASF AG, Ludwigshafen.

In general the fraction of other UV absorber is up to 10% by weight, preferably 0.001-10% by weight, in particular 0.05-10% by weight, very preferably 0.1-10% by weight, based on the total weight of the polymer composition. In the case of thin polymer layers the fraction of UV absorber used is generally higher than in the case of thick polymer layers.

The polycarbonate polymer composition may further comprise at least one 2,6-dialkylated phenol antioxidant. Suitable 2,6-dialkylated phenols are those mentioned above and, in particular, the esters of β-(3,5-di-tert-butyl-4-hydroxyphenyl)propionic acid with monohydric or polyhydric alcohols. Preferred esters of β-(3,5-di-tert-butyl-4-hydroxyphenyl)propionic acid with monohydric or polyhydric alcohols are pentaerythritol tetrakis[3-(3,5-di-tert-butyl-4-hydroxyphenyl)propionate] [CAS No. 6683-19-8], available commercially for example as Irganox® 1010 from Ciba Specialty Chemicals, Inc., octadecyl 3-(3,5-di-tert-butyl-4-hydroxyphenyl)propionate [CAS No. 2082-79-3], available commercially for example as Irganox® 1076 from Ciba Specialty Chemicals, Inc., and mixtures thereof. The fraction of antioxidant is generally up to 2000 ppm, preferably from 500 to 2000 ppm, based on the total weight of the polymer composition.

A further advantageous polymer composition comprises not only at least one 2,6-dialkylated phenol antioxidant but also at least one phosphite and/or phosphonite stabilizer. The ratio of antioxidant to costabilizer is in that case generally in the range from 1:10 to 10:1.

One advantageous polymer composition comprises at least one polycarbonate, at least one pyridinedione derivative of formula I as defined above, and, as (a) further component(s), the substance(s) indicated in a line of Table A (compositions 1.1 to 1.60). The weight fractions of the individual constituents in the compositions 1.1 to 1.60 are situated within the ranges indicated above, based on the total weight of the polymer composition.

TABLE A

| Composition | Stabilizer | UV absorber | Antioxidant |
|---|---|---|---|
| number | CAS number | CAS number | CAS number |
| 1.1 | 31570-04-4 | | |
| 1.2 | 31570-04-4 | Tinuvin ® 213 | |
| 1.3 | 31570-04-4 | 70321-86-7 | |
| 1.4 | 31570-04-4 | 3864-99-1 | |
| 1.5 | 31570-04-4 | 3147-75-9 | |
| 1.6 | 31570-04-4 | 36437-37-3 | |
| 1.7 | 31570-04-4 | 103597-45-1 | |
| 1.8 | 31570-04-4 | 147315-50-2 | |
| 1.9 | 31570-04-4 | 178671-58-4 | |
| 1.10 | 31570-04-4 | 5232-99-5 | |
| 1.11 | 31570-04-4 | | 6683-19-8 |
| 1.12 | 31570-04-4 | | 2082-79-3 |
| 1.13 | 31570-04-4 | Tinuvin ® 213 | 6683-19-8 |
| 1.14 | 31570-04-4 | 70321-86-7 | 6683-19-8 |
| 1.15 | 31570-04-4 | 3864-99-1 | 6683-19-8 |
| 1.16 | 31570-04-4 | 3147-75-9 | 6683-19-8 |
| 1.17 | 31570-04-4 | 36437-37-3 | 6683-19-8 |
| 1.18 | 31570-04-4 | 103597-45-1 | 6683-19-8 |
| 1.19 | 31570-04-4 | 147315-50-2 | 6683-19-8 |
| 1.20 | 31570-04-4 | 178671-58-4 | 6683-19-8 |
| 1.21 | 31570-04-4 | 5232-99-5 | 6683-19-8 |
| 1.22 | 31570-04-4 | Tinuvin ® 213 | 2082-79-3 |
| 1.23 | 31570-04-4 | 70321-86-7 | 2082-79-3 |
| 1.24 | 31570-04-4 | 3864-99-1 | 2082-79-3 |
| 1.25 | 31570-04-4 | 3147-75-9 | 2082-79-3 |
| 1.26 | 31570-04-4 | 36437-37-3 | 2082-79-3 |
| 1.27 | 31570-04-4 | 103597-45-1 | 2082-79-3 |
| 1.28 | 31570-04-4 | 147315-50-2 | 2082-79-3 |
| 1.29 | 31570-04-4 | 178671-58-4 | 2082-79-3 |
| 1.30 | 31570-04-4 | 5232-99-5 | 2082-79-3 |
| 1.31 | 119345-01-6 | | |
| 1.32 | 119345-01-6 | Tinuvin ® 213 | |
| 1.33 | 119345-01-6 | 70321-86-7 | |
| 1.34 | 119345-01-6 | 3864-99-1 | |
| 1.35 | 119345-01-6 | 3147-75-9 | |
| 1.36 | 119345-01-6 | 36437-37-3 | |
| 1.37 | 119345-01-6 | 103597-45-1 | |
| 1.38 | 119345-01-6 | 147315-50-2 | |
| 1.39 | 119345-01-6 | 178671-58-4 | |
| 1.40 | 119345-01-6 | 5232-99-5 | |
| 1.41 | 119345-01-6 | | 6683-19-8 |
| 1.42 | 119345-01-6 | | 2082-79-3 |
| 1.43 | 119345-01-6 | Tinuvin ® 213 | 6683-19-8 |
| 1.44 | 119345-01-6 | 70321-86-7 | 6683-19-8 |
| 1.45 | 119345-01-6 | 3864-99-1 | 6683-19-8 |
| 1.46 | 119345-01-6 | 3147-75-9 | 6683-19-8 |
| 1.47 | 119345-01-6 | 36437-37-3 | 6683-19-8 |
| 1.48 | 119345-01-6 | 103597-45-1 | 6683-19-8 |
| 1.49 | 119345-01-6 | 147315-50-2 | 6683-19-8 |
| 1.50 | 119345-01-6 | 178671-58-4 | 6683-19-8 |
| 1.51 | 119345-01-6 | 5232-99-5 | 6683-19-8 |
| 1.52 | 119345-01-6 | Tinuvin ® 213 | 2082-79-3 |
| 1.53 | 119345-01-6 | 70321-86-7 | 2082-79-3 |
| 1.54 | 119345-01-6 | 3864-99-1 | 2082-79-3 |
| 1.55 | 119345-01-6 | 3147-75-9 | 2082-79-3 |
| 1.56 | 119345-01-6 | 36437-37-3 | 2082-79-3 |
| 1.57 | 119345-01-6 | 103597-45-1 | 2082-79-3 |
| 1.58 | 119345-01-6 | 147315-50-2 | 2082-79-3 |
| 1.59 | 119345-01-6 | 178671-58-4 | 2082-79-3 |
| 1.60 | 119345-01-6 | 5232-99-5 | 2082-79-3 |

Further advantageous polymer compositions are 2.1 to 2.60, which differ from the corresponding compositions 1.1 to 1.60 only in that the polycarbonate is replaced by a polycarbonate copolymer.

Further advantageous polymer compositions are 3.1 to 3.60, which differ from the corresponding compositions 1.1 to 1.60 only in that the polycarbonate is replaced by a physical blend of polycarbonates with acrylic-butadiene-styrene-copolymers.

Further advantageous polymer compositions are 4.1 to 4.60, which differ from the corresponding compositions 1.1 to 1.60 only in that the polycarbonate is replaced by a physical blend of polycarbonates with acrylonitrile-styrene-acrylate copolymers.

Further advantageous polymer compositions are 5.1 to 5.60, which differ from the corresponding compositions 1.1 to 1.60 only in that the polycarbonate is replaced by a physical blend of polycarbonates with polymethyl methacrylates.

Further advantageous polymer compositions are 6.1 to 6.60, which differ from the corresponding compositions 1.1 to 1.60 only in that the polycarbonate is replaced by a physical blend of polycarbonates with polybutyl acrylates.

Further advantageous polymer compositions are 7.1 to 7.60, which differ from the corresponding compositions 1.1 to 1.60 only in that the polycarbonate is replaced by a physical blend of polycarbonates with polybutyl methyacrylates.

Further advantageous polymer compositions are 8.1 to 8.60, which differ from the corresponding compositions 1.1 to 1.60 only in that the polycarbonate is replaced by a physical blend of polycarbonates with poly(butylene terephthalate)s.

Further advantageous polymer compositions are 9.1 to 9.60, which differ from the corresponding compositions 1.1 to 1.60 only in that the polycarbonate is replaced by a physical blend of polycarbonates with polyethylene terephthalates.

The polycarbonate polymer composition can further comprise at least one additional component selected from the group consisting of dyes, pigments, and other additives.

Regarding suitable dyes and pigments, the remarks above are incorporated here in their entirety by reference. In one preferred embodiment the dye and/or the pigment is a bluing agent. Suitable bluing agents are, for example, ultramarine blue, phthalocyanines, anthraquinones, and indanthrones. When a bluing agent is used as well the fraction of bluing agent is up to 500 ppm (0.05% by weight), preferably 0.5-100 ppm, based on the total weight of the polymer composition.

Preferred applications for polycarbonate polymer compositions of the invention are as lenses for headlamp covers, as windshields in automobiles, and as other lens/glazing systems in automobiles and architecture.

A further advantageous composition comprises:
at least one pyridinedione derivative of formula I as defined above;
at least one polyethylene terephthalate (PET);
at least one 2,6-dialkylated phenol antioxidant;
if appropriate, at least one costabilizer selected from the group consisting of phosphites, phosphonites, and mixtures thereof; and
if appropriate, at least one further UV absorber selected from the group consisting of diphenylcyanoacrylates, phenyl-1,3,5-triazines, and benzotriazoles, and mixtures thereof.

Further advantageous is the use of at least one pyridinedione derivative of formula I as defined above in a PET composition.

Suitable 2,6-dialkylated phenol antioxidants are those mentioned above. Preference is given to the esters of β-(3,5-di-tert-butyl-4-hydroxyphenyl)propionic acid with monohydric or polyhydric alcohols and particularly pentaerythritol tetrakis[3-(3,5-di-tert-butyl-4-hydroxyphenyl)propionate] [CAS No. 6683-19-8], available commercially for example as Irganox® 1010 from Ciba Specialty Chemicals, Inc., hexamethylene-bis-(3-(3,5-di-tert-butyl-4-hydroxyphenyl)propionate [CAS No. 35074-77-2], available commercially for example as Irganox® 259 from Ciba Specialty Chemicals, Inc., and 3,5-dialkylated hydroxyphenylmethylphosphonic esters, preferably diethyl ((3,5-bis(1,1-dimethylethyl)-4-hydroxyphenyl)methyl)phosphonate [CAS No. 976-56-7], available commercially for example as Irganox® 1222 from Ciba Specialty Chemicals, Inc.

The fraction of antioxidant is generally up to 2000 ppm, preferably from 500 to 2000 ppm, based on the total weight of the polymer composition.

If appropriate, the polyethylene terephthalate polymer composition comprises at least one costabilizer selected from the group consisting of phosphites, phosphonites and mixtures thereof. Suitable phosphites and phosphonites are those mentioned above. One preferred phosphite is tris(2,4-di-tert-butylphenyl)phosphite [CAS No. 31570-04-4], available commercially for example as Irgafos® 168 from Ciba Specialty Chemicals, Inc. The fraction of phosphite and/or phosphonite is generally up to 2000 ppm, preferably from 500 to 2000 ppm, in particular from 750 to 2000 ppm, based on the total weight of the polymer composition.

One further advantageous polyethylene terephthalate polymer composition comprises not only at least one 2,6-dialkylated phenol, preferably at least one 3,5-dialkylated hydroxyphenylmethylphosphonic ester and/or at least one ester of β-(3,5-di-tert-butyl-4-hydroxyphenyl)propionic acid with monohydric or polyhydric alcohols, as antioxidant, but also at least one phosphite and/or phosphonite costabilizer. The ratio of antioxidant to costabilizer is in that case in general in the range from 1:10 to 10:1.

The polyethylene terephthalate polymer composition may further comprise at least one other UV absorber. Suitable other UV absorbers are those mentioned above. The other UV absorbers are preferably selected from the group consisting of diphenylcyanoacrylates, phenyl-1,3,5-triazines, benzotriazoles and mixtures thereof.

Examples of suitable benzotriazoles are 2-(2′-hydroxyphenyl)benzotriazoles, preferably those mentioned above. Particular preference is given to the following:
2-(2H-benzotriazol-2-yl)-4,6-bis(1-methyl-1-phenylethyl)phenol [CAS No. 70321-86-7], available commercially for example as Tinuvin® 23-4 from Ciba Specialty Chemicals, Inc.;
2,4-di-tert-butyl-6-(5-chlorobenzotriazol-2-yl)phenol [CAS No. 3864-99-1], available commercially for example as Tinuvin® 327 from Ciba Specialty Chemicals, Inc.;
2,2′-methylenebis-(6-(2H-benzotriazol-2-yl)_{4-1,1,3,3}-tetramethylbutyl)phenol [CAS No. 10359745-1], available commercially for example as Tinuvin® 360 from Ciba Specialty Chemicals, Inc.; and
transesterification products of methyl 3-(3-(2H-benzotriazol-2-yl)-5-tert-butyl-4-hydroxyphenyl)propionate with polyethylene glycol, obtainable for example as Tinuvin® 213 from Ciba Specialty Chemicals, Inc.

Examples of suitable 2-phenyl-1,3,5-triazines are 2-(2′-hydroxyphenyl)-1,3,5-triazines, preferably those mentioned above. Particular preference is given to the following:
2-(4,6-diphenyl-1,3,5-triazin-2-yl)-5-hexyloxyphenol [CAS No. 147315-50-2], available commercially for example as Tinuvin® 1577 from Ciba Specialty Chemicals, Inc.

Examples of suitable diphenylcyanoacrylates are those mentioned above. Preference is given to the following:
1,3-bis[(2′-cyano-3′,3′-diphenylacryloyl)oxy]-2,2-bis{[2′-cyano-3′,3′-diphenyl-acryloyl)oxy]methyl}propane [CAS No. 178671-58-4], available commercially for example under the name Uvinul® 3030 from BASF AG, Ludwigshafen;
ethyl 2-cyano-3,3-diphenylacrylate [CAS No. 5232-99-5], available commercially for example under the name Uvinul® 3035 from BASF AG, Ludwigshafen; and
2-ethylhexyl 2-cyano-3,3-diphenylacrylate [CAS No. 6197-30-4], obtainable commercially for example under the name Uvinul® 3039 from BASF AG, Ludwigshafen.

In general the fraction of other UV absorber is up to 2% by weight, preferably 0.01-5% by weight, in particular 0.1-0.15% by weight, based on the total weight of the polyethylene terephthalate polymer composition. In the case of thin polymer layers the fraction of UV absorber used is generally higher than in the case of thick polymer layers.

Another advantageous polyethylene terephthalate polymer composition comprises at least one pyridinedione derivative of formula I as defined above, and, as (a) further component(s), the substance(s) indicated in a line of Table B (compositions 10.1 to 10.54). The weight fractions of the individual constituents in the compositions 10.1 to 10.54 are situated within the ranges indicated above, based on the total weight of the polymer composition.

TABLE B

| Composition | Antioxidant | Costabilizer | UV Absorber |
|---|---|---|---|
| number | CAS number | CAS number | CAS number |
| 10.1 | 6683-19-8 | | |
| 10.2 | 6683-19-8 | 31570-04-4 | |
| 10.3 | 6683-19-8 | | Tinuvin ® 213 |
| 10.4 | 6683-19-8 | | 70321-86-7 |
| 10.5 | 6683-19-8 | | 3864-99-1 |
| 10.6 | 6683-19-8 | | 103597-45-1 |
| 10.7 | 6683-19-8 | | 147315-50-2 |
| 10.8 | 6683-19-8 | | 178671-58-4 |
| 10.9 | 6683-19-8 | | 5232-99-5 |
| 10.10 | 6683-19-8 | | 6197-30-4 |
| 10.11 | 6683-19-8 | 31570-04-4 | Tinuvin ® 213 |
| 10.12 | 6683-19-8 | 31570-04-4 | 70321-86-7 |
| 10.13 | 6683-19-8 | 31570-04-4 | 3864-99-1 |
| 10.14 | 6683-19-8 | 31570-04-4 | 103597-45-1 |
| 10.15 | 6683-19-8 | 31570-04-4 | 147315-50-2 |
| 10.16 | 6683-19-8 | 31570-04-4 | 178671-58-4 |
| 10.17 | 6683-19-8 | 31570-04-4 | 5232-99-5 |
| 10.18 | 6683-19-8 | 31570-04-4 | 6197-30-4 |
| 10.19 | 35074-77-2 | | |
| 10.20 | 35074-77-2 | 31570-04-4 | |
| 10.21 | 35074-77-2 | | Tinuvin ® 213 |
| 10.22 | 35074-77-2 | | 70321-86-7 |
| 10.23 | 35074-77-2 | | 3864-99-1 |
| 10.24 | 35074-77-2 | | 103597-45-1 |
| 10.25 | 35074-77-2 | | 147315-50-2 |
| 10.26 | 35074-77-2 | | 178671-58-4 |
| 10.27 | 35074-77-2 | | 5232-99-5 |
| 10.28 | 35074-77-2 | | 6197-30-4 |
| 10.29 | 35074-77-2 | 31570-04-4 | Tinuvin ® 213 |
| 10.30 | 35074-77-2 | 31570-04-4 | 70321-86-7 |
| 10.31 | 35074-77-2 | 31570-04-4 | 3864-99-1 |
| 10.32 | 35074-77-2 | 31570-04-4 | 103597-45-1 |
| 10.33 | 35074-77-2 | 31570-04-4 | 147315-50-2 |
| 10.34 | 35074-77-2 | 31570-04-4 | 178671-58-4 |
| 10.35 | 35074-77-2 | 31570-04-4 | 5232-99-5 |
| 10.36 | 35074-77-2 | 31570-04-4 | 6197-30-4 |
| 10.37 | 976-56-7 | | |
| 10.38 | 976-56-7 | 31570-04-4 | |
| 10.39 | 976-56-7 | | Tinuvin ® 213 |
| 10.40 | 976-56-7 | | 70321-86-7 |
| 10.41 | 976-56-7 | | 3864-99-1 |
| 10.42 | 976-56-7 | | 103597-45-1 |
| 10.43 | 976-56-7 | | 147315-50-2 |
| 10.44 | 976-56-7 | | 178671-58-4 |
| 10.45 | 976-56-7 | | 5232-99-5 |
| 10.46 | 976-56-7 | | 6197-30-4 |
| 10.47 | 976-56-7 | 31570-04-4 | Tinuvin ® 213 |
| 10.48 | 976-56-7 | 31570-04-4 | 70321-86-7 |
| 10.49 | 976-56-7 | 31570-04-4 | 3864-99-1 |
| 10.50 | 976-56-7 | 31570-04-4 | 103597-45-1 |
| 10.51 | 976-56-7 | 31570-04-4 | 147315-50-2 |
| 10.52 | 976-56-7 | 31570-04-4 | 178671-58-4 |
| 10.53 | 976-56-7 | 31570-04-4 | 5232-99-5 |
| 10.54 | 976-56-7 | 31570-04-4 | 6197-30-4 |

Advantageously the polyethylene terephthalate is an amorphous polyethylene terephthalate and the polyethylene terephthalate polymer composition further comprises at least one acetaldehyde scavenger. An example of a suitable acetaldehyde scavenger is anthranilamide [CAS No. 88-68-6].

Further advantageous polyethylene terephthalate polymer compositions are 11.1 to 11.54, which differ from the corresponding compositions 10.1 to 10.54 only in that the polyethylene terephthalate is an amorphous polyethylene terephthalate and the composition additionally comprises an acetaldehyde scavenger.

The polymer composition comprising the amorphous polyethylene terephthalate may additionally include at least one further component, selected from the group consisting of reheating agents, dyes, pigments, and other additives.

For the purposes of the present specification a reheating agent is a substance which by absorbing energy accelerates the plasticization of the polymer and so allows the polymer mass to be shaped by downstream assemblies (a bottle blowing mold, for example). Carbon black is an example of a suitable reheating agent. Carbon black can be used in the form of powder or granules. The fraction of reheating agent is generally from 0.1 to 2% by weight, based on the total weight of the polymer composition. Suitable dyes, pigments and other additives are those mentioned above.

Advantageous is the use of at least one pyridinedione derivative of formula I in compositions comprising an amorphous polyethylene terephthalate, at least one 2,6-dialkylated phenol antioxidant, and at least one acetaldehyde scavenger for packing materials such as bottles or containers.

Advantageously the polyethylene terephthalate is a partially crystalline polyethylene terephthalate and the polymer composition additionally comprises at least one nucleating agent. Suitable nucleating agents are those mentioned above. The fraction of nucleating agent is generally from 0.05 to 1% by weight, based on the total weight of the polymer composition.

Further advantageous polyethylene terephthalate polymer compositions are 12.1 to 12.54, which differ from the corresponding compositions 10.1 to 10.54 only in that the polyethylene terephthalate is a partially crystalline polyethylene terephthalate and the composition additionally comprises at least one nucleating agent. Areas of application for polymer compositions comprising partially crystalline polyethylene terephthalate are optical films, e.g., for displays.

Further advantageous compositions comprise:
at least one pyridinedione derivative of formula I as defined above;
at least one high-density polyethylene or one polypropylene;
at least one 2,6-dialkylated phenol antioxidant;
if appropriate, at least one costabilizer selected from the group consisting of phosphites, phosphonites, and mixtures thereof;
if appropriate, at least one further UV absorber, selected from the group consisting of diphenyl cyanoacrylates, hydroxybenzophenones, phenyl-1,3,5-triazines, benzotriazoles, and mixtures thereof;
if appropriate, at least one sterically hindered amine; and
if appropriate, a further component selected from the group consisting of dyes, pigments, and other additives.

Advantageous is the use of at least one pyridinedione derivative of formula I as defined above in a composition comprising a high-density polyethylene or a polypropylene.

Examples of suitable 2,6-dialkylated phenols are those specified above, preferably the esters of β-(3,5-di-tert-butyl-4-hydroxyphenyl)propionic acid with monohydric or polyhydric alcohols, particularly pentaerythritol tetrakis[3-(3,5-di-tert-butyl-4-hydroxyphenyl)propionate] [CAS No. 6683-19-8], available commercially for example as Irganox® 1010 from Ciba Specialty Chemicals, Inc., and octadecyl 3-(3,5-di-tert-butyl-4-hydroxyphenyl)propionate [CAS No. 2082-79-3], available commercially for example as Irganox® 1076 from Ciba Specialty Chemicals, Inc., and mixtures thereof. The fraction of antioxidant is generally up to 4000 ppm, preferably from 1000 to 4000 ppm, based on the total weight of the polymer composition.

If appropriate the composition comprises a costabilizer selected from the group consisting of phosphites, phosphonites, and mixtures thereof. Regarding suitable phosphites and phosphonites, the text above is incorporated in its entirety by reference. Preferred phosphites and phosphonites are tris(2,4-di-tert-butylohenyl) phosphite [CAS No. 31570-04-4], available commercially for example as Irgafos® 168 from Ciba Specialty Chemicals, Inc., and tetrakis(2,4-di-tert-butylphenyl) (1,1-biphenyl)-4,4′-diylbisphosphonite [CAS No. 119345-01-6], available commercially for example as Irgafos® P-EPQ from Ciba Specialty Chemicals, Inc., and mixtures thereof. The fraction of phosphite and/or phosphonite is generally up to 2000 ppm, preferably from 500 to 2000 ppm, in particular from 750 to 2000 ppm, based on the total weight of the polymer composition.

A further advantageous polymer composition comprises not only at least one 2,6-dialkylated phenol antioxidant but also at least one phosphite and/or phosphonite costabilizer. The ratio of antioxidant to costabilizer is in that case generally in the range from 1:10 to 10:1.

The polymer composition may further comprise at least one other UV absorber. Suitable other UV absorbers are those mentioned above. The other UV absorbers are preferably selected from the group consisting of diphenylcyanoacrylates, hydroxybenzophenones, phenyl-1,3,5-triazines, benzotriazoles and mixtures thereof.

Examples of suitable benzotriazoles are 2-(2′-hydroxyphenyl)benzotriazoles, preferably those mentioned above. Particular preference is given to the following:
2-(2H-benzotriazol-2-yl)-4,6-bis(1-methyl-1-phenylethyl)phenol [CAS No. 70321-86-7], available commercially for example as Tinuvin® 23-4 from Ciba Specialty Chemicals, Inc.;
2,4-di-tert-butyl-6-(5-chlorobenzotriazol-2-yl)phenol [CAS No. 3864-99-1], available commercially for example as Tinuvin® 327 from Ciba Specialty Chemicals, Inc.;
2-(2H-benzotriazol-2-yl)-4-(1,1,3,3-tetramethylbutyl)phenol [CAS No. 3147-75-9], available commercially for example as Tinuvin® 329 from Ciba Specialty Chemicals, Inc.;
2-(2H-benzotriazol-2-yl)-4-(tert-butyl)-6-(sec-butyl)phenol [CAS No. 36437-37-3], available commercially for example as Tinuvin® 350 from Ciba Specialty Chemicals, Inc.;
2,2′-methylenebis-(6-(2H-benzotriazol-2-yl)-4-1,1,3,3-tetramethylbutyl)phenol) [CAS No. 103597-45-1], available commercially for example as Tinuvin® 360 from Ciba Specialty Chemicals, Inc.; and
transesterification products of methyl 3-(3-(2H-benzotriazol-2-yl)-5-tert-butyl-4-hydroxyphenyl)propionate with polyethylene glycol, obtainable for example as Tinuvin® 213 from Ciba Specialty Chemicals, Inc.

Examples of suitable 2-phenyl-1,3,5-triazines are 2-(2′-hydroxyphenyl)-1,3,5-triazines, preferably those mentioned above. Particular preference is given to the following:
2-(4,6-diphenyl-1,3,5-triazin-2-yl)-5-hexyloxyphenol [CAS No. 147315-50-2], available commercially for example as Tinuvin® 1577 from Ciba Specialty Chemicals, Inc.; and
2,4-Bis(2,4-dimethylphenyl)-6-(2-hydroxy-4-octyloxyphenyl)-1,3,5-triazine [CAS No. 2725-22-6], available commercially for example as Cyasorb® UV 1164 from Cytec.

Examples of suitable hydroxybenzophenones are 2-hydroxybenzophenones, preferably those mentioned above. Particular preference is given to:
2-hydroxy-4-n-octoxybenzophenone [CAS No. 1843-05-6], available commercially for example as Chimassorb® 81 from Ciba Specialty Chemicals, Inc.

Examples of suitable diphenylcyanoacrylates are those mentioned above. Preference is given to the following:
1,3-bis[(2′-cyano-3′,3′-diphenylacryloyl)oxy]-2,2-bis{[2′-cyano-3′,3′-diphenyl-acryloyl)oxy]methyl}propane [CAS No. 178671-58-4], available commercially for example under the name Uvinul® 3030 from BASF AG, Ludwigshafen; and
ethyl-2-cyano-3,3-diphenylacrylate [CAS No. 5232-99-5], available commercially for example under the name Uvinul® 3035 from BASF AG, Ludwigshafen.

In general the fraction of other UV absorber is up to 2% by weight, preferably from 0.01 to 1.5% by weight, and in particular from 0.05-1% by weight, based on the total weight of the polymer composition. In the case of thin polymer layers the fraction of UV absorber used is generally higher than in the case of thick polymer layers.

The polymer composition comprising a high-density polyethylene or a polypropylene may further comprise at least one sterically hindered amine.

Suitable sterically hindered amines (HALS) are oligomeric and monomeric sterically hindered amines, examples being those mentioned above. Preferred sterically hindered amines are:
copolymers of (partially) N-piperidin-4-yl-substituted maleimide and a mixture of α-olefins, obtainable for example as Uvinul® 5050 H [CAS 152261-33-1] (molar mass approximately 3500 g/mol) of the formula 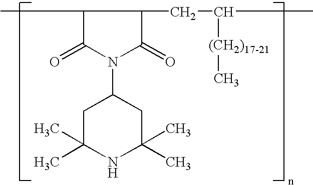
   from BASF AG, Ludwigshafen;
the sterically hindered amine of the formula [CAS No. 124172-53-8] 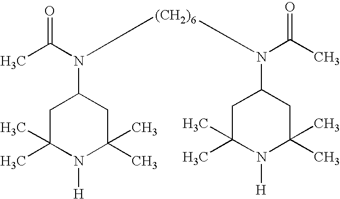
   which is obtainable for example under the name Uvinul® 4050 H from BASF AG, Ludwigshafen;
poly[[6-[(1,1,3,3-tetramethylbutyl)amino]-1,3,5-triazine-2,4-diyl][(2,2,6,6-tetramethyl-4-piperidinyl)imino]-1,6-hexanediyl[(2,2,6,6-tetramethyl-4-piperidinyl)imino]]) [CAS No. 71878-19-8], available commercially for example as Chimassorb® 944 (molar mass: 2000 to 3100 g/mol) from Ciba Specialty Chemicals, Inc.;
the dimethyl succinate polymer with 4-hydroxy-2,2,6,6-tetramethyl-1-piperidinoethanol [CAS number 65447-77-0], available commercially for example as Tinuvin® 622 (molar mass: 3100-4100 g/mol) from Ciba Specialty Chemicals, Inc.;
bis(2,2,6,6-tetramethyl-4-piperidyl)sebacate [CAS No. 52829-07-9], available commercially for example as Tinuvin® 770 from Ciba Specialty Chemicals, Inc.; and
the polymer of 2,2,4,4-tetramethyl-7-oxa-3,20-diazadispiro[5.1.11.2]heneicosan-21-one and epichlorohydrin of the formula 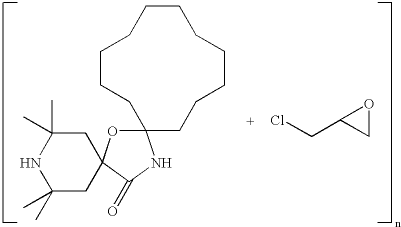
   [CAS No. 202483-55-4], available commercially for example as Hostavin® N30 from Ciba Specialty Chemicals, Inc.

In general the fraction of sterically hindered amine is up to 2% by weight, preferably 0.1-2% by weight, in particular 0.1-1.5% by weight, very preferably 0.1-1% by weight, based on the total weight of the polymer composition. In the case of thin polymer layers the fraction of sterically hindered amine used is generally higher than in the case of thick polymer layers.

The polymer composition may additionally comprise at least one further component selected from the group consisting of dyes, pigments, and other additives. Suitable dyes and pigments are those mentioned above.

Further advantageous compositions which comprise at least one high-density polyethylene or one polypropylene, at least one pyridinedione derivative of formula I as defined above, and, as (a) further component(s), the substance(s) indicated in a line of Table C (compositions 13.1 to 13.108). The weight fractions of the individual constituents in the compositions 13.1 to 13.108 are situated within the ranges indicated above, based on the total weight of the polymer composition.

TABLE C

| | | | Further light stabilizers |
|---|---|---|---|
| Composition | Antioxidant | Costabilizer | (UV absorbers/HALS) |
| number | CAS number | CAS number | CAS number |
| 13.1 | 6683-19-8 | | |
| 13.2 | 6683-19-8 | 31570-04-4 | |
| 13.3 | 6683-19-8 | | Tinuvin ® 213 |
| 13.4 | 6683-19-8 | | 70321-86-7 |
| 13.5 | 6683-19-8 | | 3864-99-1 |
| 13.6 | 6683-19-8 | | 3147-75-9 |
| 13.7 | 6683-19-8 | | 36437-37-3 |
| 13.8 | 6683-19-8 | | 103597-45-1 |
| 13.9 | 6683-19-8 | | 147315-50-2 |
| 13.10 | 6683-19-8 | | 1843-05-6 |
| 13.11 | 6683-19-8 | | 2725-22-6 |
| 13.12 | 6683-19-8 | | 178671-58-4 |
| 13.13 | 6683-19-8 | | 5232-99-5 |
| 13.14 | 6683-19-8 | | 71878-19-8 |
| 13.15 | 6683-19-8 | | 124172-53-8 |
| 13.16 | 6683-19-8 | | 152261-33-1 |
| 13.17 | 6683-19-8 | | 52829-07-9 |
| 13.18 | 6683-19-8 | | 65447-77-0 |
| 13.19 | 6683-19-8 | | 202483-55-4 |
| 13.20 | 6683-19-8 | 31570-04-4 | Tinuvin ® 213 |
| 13.21 | 6683-19-8 | 31570-04-4 | 70321-86-7 |
| 13.22 | 6683-19-8 | 31570-04-4 | 3864-99-1 |
| 13.23 | 6683-19-8 | 31570-04-4 | 3147-75-9 |
| 13.24 | 6683-19-8 | 31570-04-4 | 36437-37-3 |
| 13.25 | 6683-19-8 | 31570-04-4 | 103597-45-1 |
| 13.26 | 6683-19-8 | 31570-04-4 | 147315-50-2 |
| 13.27 | 6683-19-8 | 31570-04-4 | 1843-05-6 |
| 13.28 | 6683-19-8 | 31570-04-4 | 2725-22-6 |
| 13.29 | 6683-19-8 | 31570-04-4 | 178671-58-4 |
| 13.30 | 6683-19-8 | 31570-04-4 | 5232-99-5 |
| 13.31 | 6683-19-8 | 31570-04-4 | 71878-19-8 |
| 13.32 | 6683-19-8 | 31570-04-4 | 124172-53-8 |
| 13.33 | 6683-19-8 | 31570-04-4 | 152261-33-1 |
| 13.34 | 6683-19-8 | 31570-04-4 | 52829-07-9 |
| 13.35 | 6683-19-8 | 31570-04-4 | 65447-77-0 |
| 13.36 | 6683-19-8 | 31570-04-4 | 202483-55-4 |
| 13.37 | 6683-19-8 | 119345-01-6 | |
| 13.38 | 6683-19-8 | 119345-01-6 | Tinuvin ® 213 |
| 13.39 | 6683-19-8 | 119345-01-6 | 70321-86-7 |
| 13.40 | 6683-19-8 | 119345-01-6 | 3864-99-1 |
| 13.41 | 6683-19-8 | 119345-01-6 | 3147-75-9 |
| 13.42 | 6683-19-8 | 119345-01-6 | 36437-37-3 |
| 13.43 | 6683-19-8 | 119345-01-6 | 103597-45-1 |
| 13.44 | 6683-19-8 | 119345-01-6 | 147315-50-2 |
| 13.45 | 6683-19-8 | 119345-01-6 | 1843-05-6 |
| 13.46 | 6683-19-8 | 119345-01-6 | 2725-22-6 |
| 13.47 | 6683-19-8 | 119345-01-6 | 178671-58-4 |
| 13.48 | 6683-19-8 | 119345-01-6 | 5232-99-5 |
| 13.49 | 6683-19-8 | 119345-01-6 | 71878-19-8 |
| 13.50 | 6683-19-8 | 119345-01-6 | 124172-53-8 |
| 13.51 | 6683-19-8 | 119345-01-6 | 152261-33-1 |
| 13.52 | 6683-19-8 | 119345-01-6 | 52829-07-9 |
| 13.53 | 6683-19-8 | 119345-01-6 | 65447-77-0 |
| 13.54 | 6683-19-8 | 119345-01-6 | 202483-55-4 |
| 13.55 | 2082-79-3 | | |
| 13.56 | 2082-79-3 | 31570-04-4 | |
| 13.57 | 2082-79-3 | | Tinuvin ® 213 |
| 13.58 | 2082-79-3 | | 70321-86-7 |
| 13.59 | 2082-79-3 | | 3864-99-1 |
| 13.60 | 2082-79-3 | | 3147-75-9 |
| 13.61 | 2082-79-3 | | 36437-37-3 |
| 13.62 | 2082-79-3 | | 103597-45-1 |
| 13.63 | 2082-79-3 | | 147315-50-2 |
| 13.64 | 2082-79-3 | | 1843-05-6 |
| 13.65 | 2082-79-3 | | 2725-22-6 |
| 13.66 | 2082-79-3 | | 178671-58-4 |
| 13.67 | 2082-79-3 | | 5232-99-5 |
| 13.68 | 2082-79-3 | | 71878-19-8 |
| 13.69 | 2082-79-3 | | 124172-53-8 |
| 13.70 | 2082-79-3 | | 152261-33-1 |
| 13.71 | 2082-79-3 | | 52829-07-9 |
| 13.72 | 2082-79-3 | | 65447-77-0 |
| 13.73 | 2082-79-3 | | 202483-55-4 |
| 13.74 | 2082-79-3 | 31570-04-4 | Tinuvin ® 213 |
| 13.75 | 2082-79-3 | 31570-04-4 | 70321-86-7 |
| 13.76 | 2082-79-3 | 31570-04-4 | 3864-99-1 |
| 13.77 | 2082-79-3 | 31570-04-4 | 3147-75-9 |
| 13.78 | 2082-79-3 | 31570-04-4 | 36437-37-3 |
| 13.79 | 2082-79-3 | 31570-04-4 | 103597-45-1 |
| 13.80 | 2082-79-3 | 31570-04-4 | 147315-50-2 |
| 13.81 | 2082-79-3 | 31570-04-4 | 1843-05-6 |
| 13.82 | 2082-79-3 | 31570-04-4 | 2725-22-6 |
| 13.83 | 2082-79-3 | 31570-04-4 | 178671-58-4 |
| 13.84 | 2082-79-3 | 31570-04-4 | 5232-99-5 |
| 13.85 | 2082-79-3 | 31570-04-4 | 71878-19-8 |
| 13.86 | 2082-79-3 | 31570-04-4 | 124172-53-8 |
| 13.87 | 2082-79-3 | 31570-04-4 | 152261-33-1 |
| 13.88 | 2082-79-3 | 31570-04-4 | 52829-07-9 |
| 13.89 | 2082-79-3 | 31570-04-4 | 65447-77-0 |
| 13.90 | 2082-79-3 | 31570-04-4 | 202483-55-4 |
| 13.91 | 2082-79-3 | 119345-01-6 | |
| 13.92 | 2082-79-3 | 119345-01-6 | Tinuvin ® 213 |
| 13.93 | 2082-79-3 | 119345-01-6 | 70321-86-7 |
| 13.94 | 2082-79-3 | 119345-01-6 | 3864-99-1 |
| 13.95 | 2082-79-3 | 119345-01-6 | 3147-75-9 |
| 13.96 | 2082-79-3 | 119345-01-6 | 36437-37-3 |
| 13.97 | 2082-79-3 | 119345-01-6 | 103597-45-1 |
| 13.98 | 2082-79-3 | 119345-01-6 | 147315-50-2 |
| 13.99 | 2082-79-3 | 119345-01-6 | 1843-05-6 |
| 13.100 | 2082-79-3 | 119345-01-6 | 2725-22-6 |
| 13.101 | 2082-79-3 | 119345-01-6 | 178671-58-4 |
| 13.102 | 2082-79-3 | 119345-01-6 | 5232-99-5 |
| 13.103 | 2082-79-3 | 119345-01-6 | 71878-19-8 |
| 13.104 | 2082-79-3 | 119345-01-6 | 124172-53-8 |
| 13.105 | 2082-79-3 | 119345-01-6 | 152261-33-1 |
| 13.106 | 2082-79-3 | 119345-01-6 | 52829-07-9 |
| 13.107 | 2082-79-3 | 119345-01-6 | 65447-77-0 |
| 13.108 | 2082-79-3 | 119345-01-6 | 202483-55-4 |

A further advantage is the use of at least one pyridinedione derivative of formula I in polymer compositions comprising at least one high-density polyethylene or one polypropylene for packing materials such as bottles or containers.

Further advantageous compositions comprise:
at least one pyridinedione derivative of formula I as defined above;
at least one polystyrene;
at least one 2,6-dialkylated phenol antioxidant;
if appropriate, at least one costabilizer selected from the group consisting of phosphites, phosphonites and mixtures thereof;
if appropriate, at least one further UV absorber, selected from the group consisting of benzotriazoles, diphenylcyanoacrylates and mixtures thereof;
if appropriate, at least one sterically hindered amine; and
if appropriate, at least one further component selected from the group consisting of dyes, pigments, and other additives.

A further advantage is the use of at least one pyridinedione derivative of formula I as defined above in a polystyrene polymer composition.

Examples of suitable 2,6-dialkylated phenols are those specified above. Preferred 2,6-dialkylated phenols are the esters of β-(3,5-di-tert-butyl-4-hydroxyphenyl)propionic acid with monohydric or polyhydric alcohols, particularly pentaerythritol tetrakis[3-(3,5-di-tert-butyl-4-hydroxyphenyl)propionate] [CAS No. 6683-19-8], available commercially for example as Irganox® 1010 from Ciba Specialty Chemicals, Inc., and octadecyl 3-(3,5-di-tert-butyl-4-hydroxyphenyl)propionate [CAS No. 2082-79-3], available commercially for example as Irganox® 1076 from Ciba Specialty Chemicals, Inc., and mixtures thereof. The fraction of antioxidant is generally up to 2000 ppm, preferably from 500 to 2000 ppm, based on the total weight of the polymer composition.

Regarding suitable phosphites and phosphonites, the text above is incorporated in its entirety by reference. A preferred phosphite is tris(2,4-di-tert-butylphenyl)phosphite [CAS No. 31570-04-4], available commercially for example as Irgafos® 168 from Ciba Specialty Chemicals, Inc. The fraction of phosphite and/or phosphonite is generally up to 2000 ppm, preferably from 500 to 2000 ppm, based on the total weight of the polymer composition.

Advantageous likewise are mixtures comprising at least one 2,6-dialkylated phenol, preferably an ester of β-(3,5-di-tert-butyl-4-hydroxyphenyl)propionic acid with monohydric or polyhydric alcohols, as antioxidant, and also a phosphite and/or phosphonite as costabilizer. In that case the ratio of costabilizer to antioxidant is generally in the range from 10:1 to 1:10. Among such mixtures, particular preference is given to those comprising as costabilizer tris(2,4-di-tert-butylphenyl)phosphite [CAS No. 31570-04-4], available commercially for example as Irgafos® 168 from Ciba Specialty Chemicals, Inc., and as antioxidant pentaerythritol tetrakis[3-(3,5-di-tert-butyl-4-hydroxyphenyl)propionate] [CAS No. 6683-19-8], available commercially for example as Irganox® 1010 from Ciba Specialty Chemicals, Inc., or octadecyl 3-(3,5-di-tert-butyl-4-hydroxyphenyl)propionate [CAS No. 2082-79-3], available commercially for example as Irganox® 1076 from Ciba Specialty Chemicals, Inc. A preferred mixture is, for example, a mixture of 1 part octadecyl 3-(3,5-di-tert-butyl-4-hydroxyphenyl)propionate and 4 parts tris(2,4-di-tert-butylphenyl)phosphite, this mixture being available commercially for example as Irganox B900 from Ciba Specialty Chemicals, Inc.

The polystyrene polymer composition may further comprise at least one other UV absorber. Suitable other UV absorbers are those mentioned above. The other UV absorber is preferably selected from the group consisting of benzotriazoles, diphenylcyanoacrylates, and mixtures thereof.

Examples of suitable benzotriazoles are 2-(2′-hydroxyphenyl)benzotriazoles, preferably those mentioned above. Particular preference is given to the following:
2-(2H-benzotriazol-2-yl)-4,6-di-tert-pentylphenol [CAS No. 25973-55-1], available commercially for example as Tinuvin® 328 from Ciba Specialty Chemicals, Inc.; and
2-benzotriazol-2-yl-4-methylphenol [CAS No. 2440-22-4], available commercially for example as Tinuvin® P from Ciba Specialty Chemicals, Inc., and mixtures thereof.

Examples of suitable diphenylcyanoacrylates are those mentioned above. Preference is given to the following:
1,3-bis[(2′-cyano-3′,3′-diphenylacryloyl)oxy]-2,2-bis{[2′-cyano-3′,3′-diphenyl-acryloyl)oxy]methyl}propane [CAS No. 178671-58-4], available commercially for example under the name Uvinul® 3030 from BASF AG, Ludwigshafen; and
ethyl-2-cyano-3,3-diphenylacrylate [CAS No. 5232-99-5], available commercially for example under the name Uvinul® 3035 from BASF AG, Ludwigshafen.

In general the fraction of other UV absorber is up to 2% by weight, preferably from 0.01-1.5% by weight, and in particular from 0.05-1% by weight, based on the total weight of the polymer composition. In the case of thin polymer layers the fraction of UV absorber used is generally higher than in the case of thick polymer layers.

The polystyrene polymer composition may further comprise at least one sterically hindered amine.

Suitable sterically hindered amines are those mentioned above. The sterically hindered amine is preferably a compound of the formula:
RNH—(CH₂)₃—NR—(CH₂)₂—NR—(CH₂)₃—NHR [CAS No. 10699043-6]
where 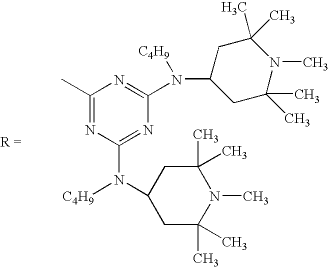
which is available commercially for example as Chimassorb® 119 from Ciba Specialty Chemicals, Inc., bis(2,2,6,6-tetramethyl-4-piperidyl) sebacate [CAS No. 52829-07-9], available commercially for example as Tinuvin® 770 from Ciba Specialty Chemicals, Inc., or mixtures thereof.

In general the fraction of sterically hindered amine is up to 2% by weight, preferably 0.1-1.5% by weight, in particular 0.1-0.5% by weight, based on the total weight of the polymer composition.

The polystyrene polymer composition may additionally comprise at least one further component selected from the group consisting of dyes, pigments, and other additives. Suitable dyes and pigments are those mentioned above.

Advantageous polystyrene polymer compositions comprise at least one pyridinedione derivative of formula I as defined above, and, as further components, the substances indicated in a line of Table D (compositions 14.1 to 14.45). The weight fractions of the individual constituents in the compositions 14.1 to 14.45 are situated within the ranges indicated above, based on the total weight of the polymer composition.

TABLE D

| | Costabilizer/ | | Sterically hindered |
|---|---|---|---|
| Composition | antioxidant | UV absorber | amine |
| No. | CAS No. | CAS No. | CAS No. |
| 14.1 | 6683-19-8 | | |
| 14.2 | 6683-19-8 | 25973-55-1 | |
| 14.3 | 6683-19-8 | 2440-22-4 | |
| 14.4 | 6683-19-8 | 178671-58-4 | |
| 14.5 | 6683-19-8 | 5232-99-5 | |
| 14.6 | 6683-19-8 | | 106990-43-6 |
| 14.7 | 6683-19-8 | | 52829-07-9 |
| 14.8 | 6683-19-8 | 25973-55-1 | 106990-43-6 |
| 14.9 | 6683-19-8 | 2440-22-4 | 106990-43-6 |
| 14.10 | 6683-19-8 | 178671-58-4 | 106990-43-6 |
| 14.11 | 6683-19-8 | 5232-99-5 | 106990-43-6 |
| 14.12 | 6683-19-8 | 25973-55-1 | 52829-07-9 |
| 14.13 | 6683-19-8 | 2440-22-4 | 52829-07-9 |
| 14.14 | 6683-19-8 | 178671-58-4 | 52829-07-9 |
| 14.15 | 6683-19-8 | 5232-99-5 | 52829-07-9 |
| 14.16 | 2082-79-3 | | |
| 14.17 | 2082-79-3 | 25973-55-1 | |
| 14.18 | 2082-79-3 | 2440-22-4 | |
| 14.19 | 2082-79-3 | 178671-58-4 | |
| 14.20 | 2082-79-3 | 5232-99-5 | |
| 14.21 | 2082-79-3 | | 106990-43-6 |
| 14.22 | 2082-79-3 | | 52829-07-9 |
| 14.23 | 2082-79-3 | 25973-55-1 | 106990-43-6 |
| 14.24 | 2082-79-3 | 2440-22-4 | 106990-43-6 |
| 14.25 | 2082-79-3 | 178671-58-4 | 106990-43-6 |
| 14.26 | 2082-79-3 | 5232-99-5 | 106990-43-6 |
| 14.27 | 2082-79-3 | 25973-55-1 | 52829-07-9 |
| 14.28 | 2082-79-3 | 2440-22-4 | 52829-07-9 |
| 14.29 | 2082-79-3 | 178671-58-4 | 52829-07-9 |
| 14.30 | 2082-79-3 | 5232-99-5 | 52829-07-9 |
| 14.31 | 1 part 2082-79-3 and | | |
| | 4 parts 31570-04-4 | | |
| 14.32 | 1 part 2082-79-3 and | 25973-55-1 | |
| | 4 parts 31570-04-4 | | |
| 14.33 | 1 part 2082-79-3 and | 2440-22-4 | |
| | 4 parts 31570-04-4 | | |
| 14.34 | 1 part 2082-79-3 and | 178671-58-4 | |
| | 4 parts 31570-04-4 | | |
| 14.35 | 1 part 2082-79-3 and | 5232-99-5 | |
| | 4 parts 31570-04-4 | | |
| 14.36 | 1 part 2082-79-3 and | | 106990-43-6 |
| | 4 parts 31570-04-4 | | |
| 14.37 | 1 part 2082-79-3 and | | 52829-07-9 |
| | 4 parts 31570-04-4 | | |
| 14.38 | 1 part 2082-79-3 and | 25973-55-1 | 106990-43-6 |
| | 4 parts 31570-04-4 | | |
| 14.39 | 1 part 2082-79-3 and | 2440-22-4 | 106990-43-6 |
| | 4 parts 31570-04-4 | | |
| 14.40 | 1 part 2082-79-3 and | 178671-58-4 | 106990-43-6 |
| | 4 parts 31570-04-4 | | |
| 14.41 | 1 part 2082-79-3 and | 5232-99-5 | 106990-43-6 |
| | 4 parts 31570-04-4 | | |
| 14.42 | 1 part 2082-79-3 and | 25973-55-1 | 52829-07-9 |
| | 4 parts 31570-04-4 | | |
| 14.43 | 1 part 2082-79-3 and | 2440-22-4 | 52829-07-9 |
| | 4 parts 31570-04-4 | | |
| 14.44 | 1 part 2082-79-3 and | 178671-58-4 | 52829-07-9 |
| | 4 parts 31570-04-4 | | |
| 14.45 | 1 part 2082-79-3 and | 5232-99-5 | 52829-07-9 |
| | 4 parts 31570-04-4 | | |

Advantageous is the use of at least one pyridinedione derivative of formula I in polystyrene polymer compositions for packaging such as yogurt pots and casings of electrical instruments.

Further advantageous compositions comprise:
at least one pyridinedione derivative of formula I as defined above;
at least one acrylonitrile-butadiene-styrene copolymer or styrene-acrylonitrile copolymer;
at least one 2,6-dialkylated phenol antioxidant;
if appropriate, at least one costabilizer selected from the group consisting of phosphites, phosphonites, and mixtures thereof;
if appropriate, at least one further UV absorber, selected from the group consisting of benzotriazoles, hydroxybenzophenones, diphenylcyanoacrylates, and mixtures thereof;
if appropriate, at least one sterically hindered amine; and
if appropriate, a further component selected from the group consisting of dyes, pigments, and other additives.

Advantageous is the use of at least one pyridinedione derivative of formula I as defined above in an acrylonitrile-butadiene-styrene copolymer or styrene-acrylonitrile copolymer composition.

Examples of suitable 2,6-dialkylated phenols are the esters of β-(3,5-di-tert-butyl-4-hydroxyphenyl)propionic acid with monohydric or polyhydric alcohols and in particular octadecyl 3-(3,5-di-tert-butyl-4-hydroxyphenyl)propionate [CAS No. 2082-79-3], available commercially for example as Irganox® 1076 from Ciba Specialty Chemicals, Inc. The fraction of antioxidant is generally up to 2000 ppm, preferably from 500 to 2000 ppm, based on the total weight of the polymer composition.

Regarding suitable phosphites and phosphonites, the text above is incorporated in its entirety by reference. Preferred phosphites and phosphonites are tris(2,4-di-tert-butylphenyl)phosphite [CAS No. 31570-04-4], available commercially for example as Irgafos® 168 from Ciba Specialty Chemicals, Inc., and tetrakis(2,4-di-tert-butylphenyl)[1,1-b]phenyl]-4,4′-diylbisphosphonite [CAS No. 119345-01-6], available commercially for example as Irgafos® P-EPQ from Ciba Specialty Chemicals, Inc., and mixtures thereof. The fraction of phosphite and/or phosphonite is generally up to 2000 ppm, preferably from 500 to 2000 ppm, based on the total weight of the polymer composition.

Advantageous polymer compositions comprise not only at least one 2,6-dialkylated phenol, preferably an ester of β-(3,5-di-tert-butyl-4-hydroxyphenyl)propionic acid with monohydric or polyhydric alcohols, as antioxidant, but also at least one phosphite and/or phosphonite costabilizer. In that case the ratio of antioxidant to costabilizer is generally in the range from 1:10 to 10:1.

The polymer composition which comprises at least one acrylonitrile-butadiene-styrene copolymer or one styrene-acrylonitrile copolymer may additionally comprise at least one further UV absorber. Suitable other UV absorbers are those mentioned above. The other UV absorbers are preferably selected from the group consisting of benzotriazoles, hydroxybenzophenones, diphenylcyanoacrylates, and mixtures thereof.

Examples of suitable benzotriazoles are 2-(2′-hydroxyphenyl)benzotriazoles, preferably those mentioned above. Particular preference is given to the following:
2,4-di-tert-butyl-6-(5-chlorobenzotriazol-2-yl)phenol [CAS No. 3864-99-1], available commercially for example as Tinuvin® 327 from Ciba Specialty Chemicals, Inc.,
2-benzotriazol-2-yl-4-methylphenol [CAS No. 2440-22-4], available commercially for example as Tinuvin® P from Ciba Specialty Chemicals, Inc.

Examples of suitable hydroxybenzophenones are 2-hydroxybenzophenones. Particular preference is given to:
2-hydroxy-4-n-octoxybenzophenone [CAS No. 1843-05-6], available commercially for example as Chimassorb® 81 from Ciba Specialty Chemicals, Inc.

Examples of suitable diphenylcyanoacrylates are:
1,3-bis[(2′-cyano-3′,3′-diphenylacryloyl)oxy]-2,2-bis{[2′-cyano-3′,3′-diphenyl-acryloyl)oxy]methyl}propane [CAS No. 178671-58-4], available commercially for example under the name Uvinul® 3030 from BASF AG, Ludwigshafen; and
ethyl-2-cyano-3,3-diphenylacrylate [CAS No. 5232-99-5], available commercially for example under the name Uvinul® 3035 from BASF AG, Ludwigshafen.

In general the fraction of other UV absorber is up to 2% by weight, preferably from 0.01-1.5% by weight, and in particular from 0.05-1% by weight, based on the total weight of the polymer composition. In the case of thin polymer layers the fraction of UV absorber used is generally higher than in the case of thick polymer layers.

The acrylonitrile-butadiene-styrene copolymer or styrene-acrylonitrile copolymer composition may further comprise at least one sterically hindered amine. Suitable sterically hindered amines are those mentioned above. The sterically hindered amine is preferably a compound of the formula:
RNH—(CH₂)₃—NR—(CH₂)₂—NR—(CH₂)₃—NHR [CAS No. 10699043-6]
where 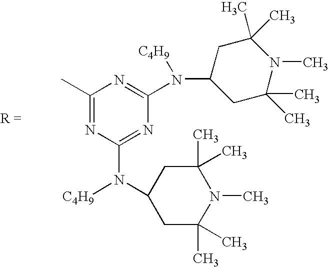
which is available commercially for example as Chimassorb® 119 from Ciba Specialty Chemicals, Inc., bis(2,2,6,6-tetramethyl-4-piperidyl)sebacate [CAS No. 52829-07-9], available commercially for example as Tinuvin® 770 from Ciba Specialty Chemicals, Inc., or mixtures thereof.

In general the fraction of sterically hindered amine is up to 2% by weight, preferably 0.1-1.5% by weight, in particular 0.1-1% by weight, very preferably 0.1-0.5% by weight, based on the total weight of the polymer composition.

The acrylonitrile-butadiene-styrene copolymer or styrene-acrylonitrile copolymer composition may additionally comprise at least one further component selected from the group consisting of dyes, pigments, and other additives. Suitable dyes and pigments are those mentioned above.

Advantageous acrylonitrile-butadiene-styrene copolymer or styrene-acrylonitrile copolymer compositions comprise at least one pyridinedione derivative of formula I as defined above, and, as further components, the substances indicated in a line of Table E (compositions 15.1 to 15.54). The weight fractions of the individual constituents in the compositions 15.1 to 15.54 are situated within the ranges indicated above, based on the total weight of the polymer composition.

TABLE E

| | | | Sterically |
|---|---|---|---|
| Compo- | Antioxidant/costabilizer | UV absorber | hindered |
| sition No. | CAS No. | CAS No. | amine CAS No. |
| 15.1 | 2082-79-3 | | |
| 15.2 | 2082-79-3 and 31570-04-4 | | |
| 15.3 | 2082-79-3 | 3864-99-1 | |
| 15.4 | 2082-79-3 | 1843-05-6 | |
| 15.5 | 2082-79-3 | 2440-22-2 | |
| 15.6 | 2082-79-3 | 178671-58-4 | |
| 15.7 | 2082-79-3 | 5232-99-5 | |
| 15.8 | 2082-79-3 | | 106990-43-6 |
| 15.9 | 2082-79-3 | | 52829-07-9 |
| 15.10 | 2082-79-3 | 3864-99-1 | 106990-43-6 |
| 15.11 | 2082-79-3 | 1843-05-6 | 106990-43-6 |
| 15.12 | 2082-79-3 | 2440-22-2 | 106990-43-6 |
| 15.13 | 2082-79-3 | 178671-58-4 | 106990-43-6 |
| 15.14 | 2082-79-3 | 5232-99-5 | 106990-43-6 |
| 15.15 | 2082-79-3 | 3864-99-1 | 52829-07-9 |
| 15.16 | 2082-79-3 | 1843-05-6 | 52829-07-9 |
| 15.17 | 2082-79-3 | 2440-22-2 | 52829-07-9 |
| 15.18 | 2082-79-3 | 178671-58-4 | 52829-07-9 |
| 15.19 | 2082-79-3 | 5232-99-5 | 52829-07-9 |
| 15.20 | 2082-79-3 and 31570-04-4 | 3864-99-1 | 106990-43-6 |
| 15.21 | 2082-79-3 and 31570-04-4 | 1843-05-6 | 106990-43-6 |
| 15.22 | 2082-79-3 and 31570-04-4 | 2440-22-2 | 106990-43-6 |
| 15.23 | 2082-79-3 and 31570-04-4 | 178671-58-4 | 106990-43-6 |
| 15.24 | 2082-79-3 and 31570-04-4 | 5232-99-5 | 106990-43-6 |
| 15.25 | 2082-79-3 and 31570-04-4 | 3864-99-1 | 52829-07-9 |
| 15.26 | 2082-79-3 and 31570-04-4 | 1843-05-6 | 52829-07-9 |
| 15.27 | 2082-79-3 and 31570-04-4 | 2440-22-2 | 52829-07-9 |
| 15.28 | 2082-79-3 and 31570-04-4 | 178671-58-4 | 52829-07-9 |
| 15.29 | 2082-79-3 and 31570-04-4 | 5232-99-5 | 52829-07-9 |
| 15.30 | 2082-79-3 and 119345-01-6 | | |
| 15.31 | 2082-79-3 and 119345-01-6 | 3864-99-1 | 106990-43-6 |
| 15.32 | 2082-79-3 and 119345-01-6 | 1843-05-6 | 106990-43-6 |
| 15.33 | 2082-79-3 and 119345-01-6 | 2440-22-2 | 106990-43-6 |
| 15.34 | 2082-79-3 and 119345-01-6 | 178671-58-4 | 106990-43-6 |
| 15.35 | 2082-79-3 and 119345-01-6 | 5232-99-5 | 106990-43-6 |
| 15.36 | 2082-79-3 and 119345-01-6 | 3864-99-1 | 52829-07-9 |
| 15.37 | 2082-79-3 and 119345-01-6 | 1843-05-6 | 52829-07-9 |
| 15.38 | 2082-79-3 and 119345-01-6 | 2440-22-2 | 52829-07-9 |
| 15.39 | 2082-79-3 and 119345-01-6 | 178671-58-4 | 52829-07-9 |
| 15.40 | 2082-79-3 and 119345-01-6 | 5232-99-5 | 52829-07-9 |
| 15.41 | 2082-79-3 and 31570-04-4 | 3864-99-1 | |
| 15.42 | 2082-79-3 and 31570-04-4 | 1843-05-6 | |
| 15.43 | 2082-79-3 and 31570-04-4 | 2440-22-2 | |
| 15.44 | 2082-79-3 and 31570-04-4 | 178671-58-4 | |
| 15.45 | 2082-79-3 and 31570-04-4 | 5232-99-5 | |
| 15.46 | 2082-79-3 and 31570-04-4 | | 106990-43-6 |
| 15.47 | 2082-79-3 and 31570-04-4 | | 52829-07-9 |
| 15.48 | 2082-79-3 and 119345-01-6 | 3864-99-1 | |
| 15.49 | 2082-79-3 and 119345-01-6 | 1843-05-6 | |
| 15.50 | 2082-79-3 and 119345-01-6 | 2440-22-2 | |
| 15.51 | 2082-79-3 and 119345-01-6 | 178671-58-4 | |
| 15.52 | 2082-79-3 and 119345-01-6 | 5232-99-5 | |
| 15.53 | 2082-79-3 and 119345-01-6 | | 106990-43-6 |
| 15.54 | 2082-79-3 and 119345-01-6 | | 52829-07-9 |

Advantageous is the use of at least one pyridinedione derivative of formula I in acrylonitrile-butadiene-styrene copolymer or styrene-acrylonitrile copolymer compositions used in components for automobiles and casings of electrical instruments.

The pyridinedione derivative of formula I for use in accordance with the invention and, where present, the compounds of groups a) to s) and/or the other additives of group t) are added to the plastic. Addition is made in a customary way, by blending with the plastic, for example. Thus the pyridinedione derivatives and, if appropriate, the further stabilizers may also be added to the starting monomers, and the mixture of monomers and stabilizers can be polymerized. It is also possible to add the pyridinedione derivatives and, if appropriate, the compounds of groups a) to s) and/or the other additives of group t) to the monomers during the polymerization. A precondition for addition before or during polymerization is that the pyridinedione derivatives and, if appropriate, the compounds of groups a) to s) and/or the other additives of group t) are stable under the polymerization conditions: that is, that they exhibit little or no decomposition.

It is preferred to add the pyridinedione derivatives and, if appropriate, the compounds of groups a) to s) and/or the other additives of group t) to the finished plastic. This is effected in a usual fashion by mixing methods which are known per se: for example, with melting at temperatures from 150 to 300° C. However, the components can also be mixed “cold”, without melting, and the pulverulent or granular mixture is not melted and homogenized until during processing.

It will be appreciated that the pyridinedione derivatives of formula I and, if appropriate, the compounds of groups a) to s) and/or the other additives of group t) can be added together or separately from one another, all at once, in portions, or continuously, at a constant rate or along a gradient. For example, part of the pyridinedione derivatives can be added to the monomers during the actual polymerization, and the remainder can be added only to the finished polymer, or all of the pyridinedione derivative can be added to the finished polymer.

Blending takes place preferably in a customary extruder, with the components being able to be introduced into the extruder as a mixture or individually, completely for example, by way of a hopper or else introduced proportionally at a later point in the extruder to the melt or solid product in the extruder. For melt extrusion particular suitability is possessed by, for example, single-screw or twin-screw extruders. A twin-screw extruder is preferred.

The mixtures obtained can be pelletized or granulated, for example, or processed by methods which are common knowledge: for example, by extrusion, injection molding, foaming with blowing agents, thermoforming, hollow body blowing or calendering.

The plastics can preferably be used to produce moldings (including semifinished products, films, sheets, and foams) of all kinds, examples being packaging and films, for textiles for example, particularly packaging for cosmetics, perfumes, and pharmaceutical products, and packaging and films for foods, beverage bottles, or packaging for cleaning products. It is also possible to produce stretch films from thermoplastic molding compounds.

Any product may in principle be protected by a packaging which comprises the pyridinedione derivatives of formula I. The product to be protected is preferably selected from cosmetics, drugs, perfumes, foods, and cleaning products. Suitable cosmetics include soap, body lotion, skin cream, shower gel, bubble bath, body spray, makeup, eyeliner, mascara, rouge, lipstick, shampoo, hair conditioner, hair gel, hair wax, hair lotion, nail varnish, nail varnish remover, etc. Suitable pharmaceutical products include pharmaceutical compositions or drugs in the form of tablets, pills, film-coated tablets, suppositories, solutions, concentrates, suspensions, and the like. Suitable foods include carbonated and noncarbonated beverages, examples being carbonated beverages such as lemonade, beer, carbonated fruit juice drinks, carbonated water, noncarbonated beverages such as wine, fruit juice, tea or coffee, fruit, meat, sausage, dairy products such as milk, yogurt, butter or cheese, animal and vegetable fats, bakery products, pasta, seasonings, sauces, pastes, pestos, stocks, purees, ketchups, dressings, etc. Suitable cleaning products include household cleaners and industrial cleaners.

The pyridinedione derivatives of formula I are used with particular preference in thermoplastic molding compounds comprising polyolefins for agricultural films and packaging films, in biaxially oriented polypropylene for stretch-wrap films, in polyethylene terephthalate or polyethylene naphthalate for bottles and other container packs, in polyvinyl butyral for laminated glass, in polystyrene for blister packs and other packaging containers, in polycarbonate for bottles, flasks and other packaging containers and moldings, in polyvinyl chloride for packaging containers and films, or in polyvinyl alcohol for producing films.

If appropriate, the films of different polymers can be combined with one another by lamination or in the form of extrusion laminates to form composite films.

By monoaxial or biaxial stretching it is possible in general to improve the properties of films. This is utilized, for example, in order to produce shrink films. Shrink films can be produced from polyethylene terephthalate, polyethylene, polyvinylidene chloride or polyvinyl chloride, for example.

The materials stabilized using at least one pyridinedione derivative of formula I exhibit particular quality features in comparison to unstabilized materials and to materials stabilized with prior art stabilizers. The materials stabilized in accordance with the invention feature an extended exposure time, since light-induced damage does not begin until later. Moreover, the material stabilized using at least one pyridinedione derivative of formula I protects not only the material to be stabilized but also the packaged contents.

The present invention further provides pyridinedione derivatives of general formula I and if appropriate tautomers thereof and also the preferred embodiments thereof, which have already been set out above in connection with their inventive use.

Where R² in general formula I corresponds to a group NR⁴R⁵ the radicals R⁴ and R⁵ are preferably different.

Preferably, moreover, R⁴ independently of R¹ has the definition of R¹, and R⁵ the definition of COR⁶.

R⁶ in that case corresponds in particular to aryl or heteroaryl which is unsubstituted or carries one or more radicals selected independently of one another from the group consisting of C₁-C₁₈ alkyl, C₁-C₆ alkoxy, cyano, CONZ²Z³ and CO₂Z⁴, preferably phenyl which is unsubstituted or carries one or more radicals selected independently of one another from the group consisting of C₁-C₆ alkyl, C₁-C₆ alkoxy, cyano, CONZ²Z³ and CO₂Z⁴, more preferably phenyl which is unsubstituted or carries one or more radicals selected independently of one another from the group consisting of C₁-C₄ alkyl, C₁-C₄ alkoxy and cyano. The variables Z², Z³ and Z⁴ here correspond to the definition already given earlier on above.

Further of particular interest are compounds of formulae 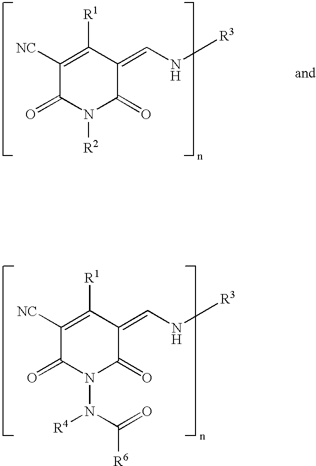
in which
R¹ and R² independently of one another are hydrogen, C₁-C₄ alkyl, partially fluorinated or perfluorinated C₁-C₄ alkyl,
   or
   phenyl which is unsubstituted or carries one or more radicals selected independently of one another from the group consisting of C₁-C₄ alkyl, C₁-C₄ alkoxy and cyano,
R⁴ and R⁶ independently of one another and of R¹ and R² respectively have the definition of R¹ and R² respectively,
n denotes values of 1, 2, 3 or 4
   and
R³ if n is 1
   is hydrogen,
   C₁-C₈ alkyl whose carbon chain may be interrupted by one group —O— and/or which is optionally substituted one or more times by identical or different radicals selected from the group consisting of hydroxyl, fluoro, carboxyl, C₁-C₄ alkoxycarbonyl, C₁-C₄ alkanoyloxy and aryl, with the aryl group being unsubstituted or carrying one or more substitutents selected independently of one another from the group consisting of C₁-C₄ alkyl and C₁-C₄ alkoxy,
   cyclopentyl or cyclohexyl which are unsubstituted or carry one or more C₁-C₄ alkyl groups,
   or
   piperidyl which is unsubstituted or carries one or more C₁-C₄ alkyl groups,
   if n is not 1
   is n-valent C₂-C₁₂ alkyl, n-valent cyclopentyl, n-valent cyclohexyl or n-valent piperidyl, which is unsubstituted or carries one or more C₁-C₄ alkyl groups.

Mention may be made in particular of compounds shown below.

If n is 1: 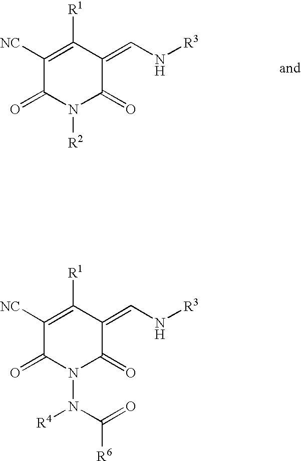
in which the variables are as defined above.

If n is 2: 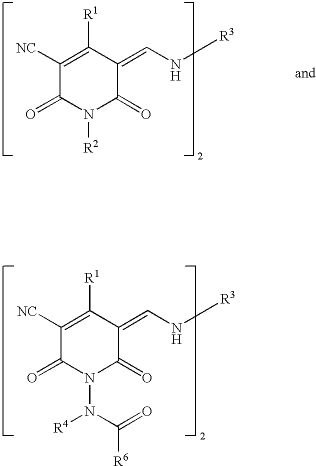
in which
R¹ and R² independently of one another are hydrogen, C₁-C₄ alkyl, partially fluorinated or perfluorinated C₁-C₄ alkyl,
   or
   phenyl which is unsubstituted or carries one or more radicals selected independently of one another from the group consisting of C₁-C₄ alkyl, C₁-C₄ alkoxy and cyano,
R⁴ and R⁶ independently of one another and of R¹ and R² respectively have the definition of R¹ and R² respectively
   and
R³ is C₂-C₁₂ alkylene.

If n is 3: 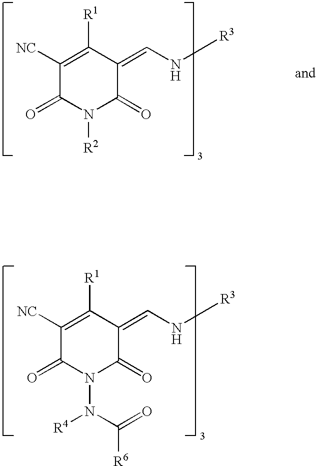
in which
R¹ and R² independently of one another are hydrogen, C₁-C₄ alkyl, partially fluorinated or perfluorinated C₁-C₄ alkyl,
   or
   phenyl which is unsubstituted or carries one or more radicals selected independently of one another from the group consisting of C₁-C₄ alkyl, C₁-C₄ alkoxy and cyano,
R⁴ and R⁶ independently of one another and of R¹ and R² respectively have the definition of R¹ and R² respectively
   and
R³ is trivalent C₃-C₁₂ alkyl.

Examples of trivalent alkyl radicals are

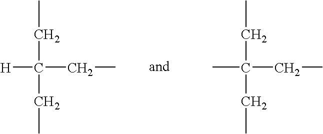

If n is 4: 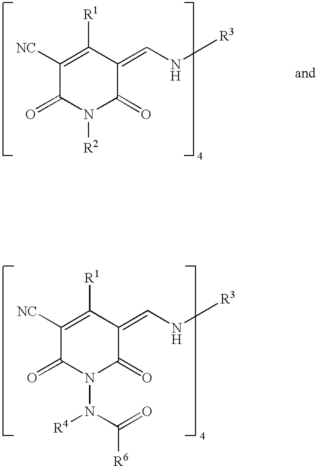
in which
R¹ and R² independently of one another are hydrogen, C₁-C₄ alkyl, partially fluorinated or perfluorinated C₁-C₄ alkyl,
   or
   phenyl which is unsubstituted or carries one or more radicals selected independently of one another from the group consisting of C₁-C₄ alkyl, C₁-C₄ alkoxy and cyano,
R⁴ and R⁶ independently of one another and of R¹ and R² respectively have the definition of R¹ and R² respectively
   and
R³ is tetravalent C₄-C₁₂ alkyl.

Examples of tetravalent alkyl radicals are

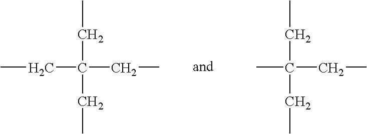

If n adopts values of 2, 3 or 4, the fragments 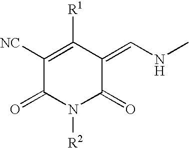
are preferably attached to different carbon atoms of the n-valent radical R³. In order to ensure this a C₂, C₃ or C₄ alkyl chain has been taken as the minimum chain length of the n-valent alkyl radical R³ for n as 2, 3 or 4 respectively.

Although it is also possible in principle for two or three such fragments to be attached to the same carbon atom of the n-valent alkyl radical, it is nevertheless expected that such pyridinedione derivatives will generally lack sufficient stability to hydrolysis.

The examples below are intended to illustrate the invention, though without restricting it.

### I. PREPARATION EXAMPLES

The pyridinedione derivatives were synthesized in accordance with the two-stage route already addressed above:

A) Preparation of the 5-dimethylaminomethylene-Substituted Intermediate

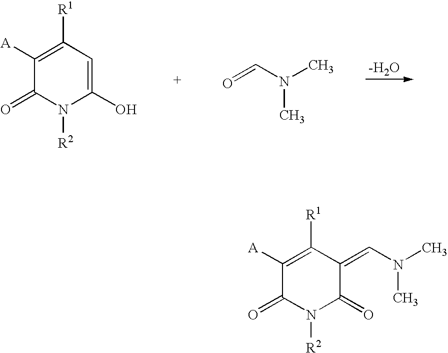

B) Reaction of the Intermediate with the N-Functional Amine 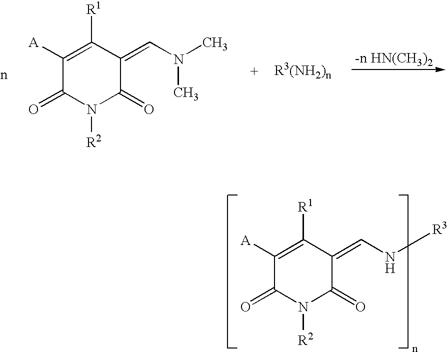
to give the target compound.

### Example 1

### A) Preparation of 1,4-dimethyl-5-dimethylaminomethylene-2,6-dioxo-3-cyano-1,2,5,6-tetrahydropyridine

78.0 g (475 mmol) of 1,4-dimethyl-6-hydroxy-3-cyano-2-pyridone and 52.1 g (713 mmol) of dimethylformamide are heated at 80° C. in acetic anhydride (360 ml). The reaction mixture is held at this temperature for 1.5 h and then cooled to room temperature. The product is isolated by filtration and washed with acetic anhydride and ether. This gives 97.7 g (94%) of 1,4-dimethyl-5-dimethylaminomethylene-2,6-dioxo-3-cyano-1,2,5,6-tetrahydropyridine.

¹H NMR (d6-DMSO, 500 MHz): 2.34 (s, 3H); 3.11 (s, 3H); 3.55 (s, 3H), 8.36 (s, 1H).

UV (acetonitrile): λₘₐₓ(Ig ε) 374 nm (4.52).

### B) Preparation of 5-butylaminomethylene-1,4-dimethyl-2,6-dioxo-3-cyano-1,2,5,6-tetrahydropyridine

5.00 g (22.8 mmol) of 1,4-dimethyl-5-dimethylaminomethylene-2,6-dioxo-3-cyano-1,2,5,6-tetrahydropyridine are suspended in ethanol (115 ml) and 1.67 g (22.8 mmol) of butylamine are added. The mixture is heated under reflux for 4 h and then cooled to room temperature. The product is isolated by filtration and washed with ether. This gives 3.70 g (65%) of 5-butylaminomethylene-1,4-dimethyl-2,6-dioxo-3-cyano-1,2,5,6-tetrahydropyridine as a pale yellow powder.

m.p.: 176-177° C.

¹H NMR (CDCl₃, 500 MHz): 0.97 (t, J=7.5 Hz, 3H); 1.39-1.47 (m, 2H), 1.70-1.73 (m, 2H); 2.42 (s, 3H); 3.30 (s, 3H); 3.55 (q, J=7.0 Hz, 2H); 7.79 (d, 1H, J=13.5 Hz, 1H); 11.41 (br s, 1H).

UV (acetonitrile): λₘₐₓ(Ig ε) 358 nm (4.61).

### Examples 2-22 and C1 and C2 (Comparative)

The other aminomethylene-substituted pyridinediones were prepared in the same way as for Example 1, by synthesis of the intermediate compound according to stage A) and reaction of the intermediate according to stage B), using the corresponding amines R³NH₂ (n=1) or, in the case of Examples 20 to 22, using hexamethylenediamine (n=2). Furthermore, in the same way, comparison compounds C1 and C2 were prepared using the corresponding aromatic amines R³NH₂ (R³ being C₆H₅ and o,o′-di-iso-C₃H₇—C₆H₃). The melting points and spectroscopic properties of the corresponding pyridinedione derivatives and also of comparison compounds C1 and C2 are summarized in Tables 1a and 1b and also 1c.

TABLE 1a
(variable A in formula I in all cases CN)

| | | | | m.p. | λₘₐₓ | |
|---|---|---|---|---|---|---|
| Ex. | R¹ | R² | R³ | [° C.] | [nm] | Ig ε |
| 1 | CH₃ | CH₃ | n-C₄H₉ | 200 | 358 | 4.61 |
| 2 | CH₃ | CH₃ | CH₂-tert-C₄H₉ | 200 | 358 | 4.62 |
| 3 | CH₃ | CH₃ | tert-C₄H₉ | 256 | 358 | 4.61 |
| 4 | CH₃ | CH₃ | H | 311 | 352 | 4.52 |
| 5 | CH₃ | NHCOC₆H₅ | n-C₄H₉ | 218 | 362 | 4.60 |
| 6 | CH₃ | NHCOC₆H₅ | CH₂-tert-C₄H₉ | 309 | 364 | 4.61 |
| 7 | CF₃ | CH₃ | n-C₄H₉ | 102 | 378 | 4.59 |
| 8 | CF₃ | CH₃ | CH₂-tert-C₄H₉ | 90 | 376 | 4.62 |
| 9 | CH₃ | CH₃ | CH(CH₃)—C₆H₅ | 141 | 366 | 4.62 |
| 10 | CH₃ | CH₃ | iso-C₃H₇ | 200 | 358 | 4.61 |
| 11 | CH₃ | CH₃ | CH₂CH₂OH | 243 | 360 | 4.62 |
| 12 | CH₃ | CH₃ | CH₂CH₂OCH₃ | 186 | 358 | 4.61 |
| 13 | CH₃ | CH₃ | CH₂CH₂OOCCH₃ | 187 | 362 | 4.55 |
| 14 | CH₃ | CH₃ | cyc-C₆H₁₁ | 165 | 364 | 4.62 |
| 15 | CH₃ | CH₃ | cyc-C₅H₉ | 183 | 364 | 4.62 |
| 16 | CH₃ | CH₃ | CH₂—C₆H₅ | 167 | 362 | 4.43 |
| 17 | CH₃ | CH₃ | 2-ethylhexyl | 90 | 364 | 4.61 |
| 18 | C₆H₅ | CH₃ | CH(CH₃)—C₆H₅ | 148 | 364 | 4.62 |
| 19 | C₆H₅ | CH₃ | CH₂-tert-C₄H₉ | 200 | 362 | 4.60 |

TABLE 1b

| | | | m.p. | λₘₐₓ | |
|---|---|---|---|---|---|
| Ex. | | R¹ | [° C.] | [nm] | lg ε |
| 20 21 22 | 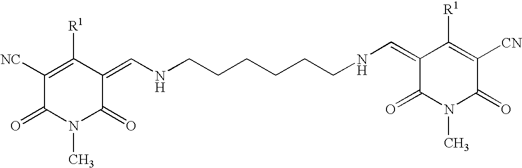 | CH₃CF₃C₆H₅ | 288 159 244 | 360 380 362 | 4.89 4.70 4.86 |

TABLE 1c
(variable A in formula I in both cases CN)

| | | | | m.p. | λₘₐₓ | |
|---|---|---|---|---|---|---|
| Ex. | R¹ | R² | R³ | [° C.] | [nm] | Ig ε |
| C1 | CH₃ | CH₃ | C₆H₅ | 268 | 384 | 4.55 |
| C2 | CH₃ | CH₃ | o,o′-di-iso-C₃H₇—C₆H₃ | 193 | 388 | 4.56 |

### II. USE EXAMPLES

### Examples 23-34 and C3 and C4 (Comparative)

### Incorporation of the Pyridinedione Derivatives into Polyethylene Terephthalate (PET)

A mixture of a polyethylene terephthalate (Polyclear T94 from Ter Hell & Co GmbH, Hamburg) and 200 to 5000 ppm (by weight) of the pyridinedione derivative or 2000 ppm (by weight) of comparison compounds C1 and C2 were homogenized in a Berstorff twin-screw extruder (melt temperature: 275° C.) and then granulated. The resultant granules were subsequently extruded in a Weber single-screw extruder through a slot die (melt temperature: 225° C.) and pressed to a thickness of 300 μm via a roll takeoff.

The results are summarized in tables 2a and 2b. The parameters reported are the wavelengths below which less than 10% or 20% of the radiation passes through the film. A value below and preferably close to 400 nm signifies that the material beneath the film is effectively protected against UV radiation.

Also reported is the yellow value in the form of the Yellowness Index (YI, measured in accordance with DIN 6167). A low value denotes low yellowing of the polymer.

TABLE 2a

| | | | Wavelength | Wavelength | |
|---|---|---|---|---|---|
| | Compound | Concentration | 10%* | 20%* | |
| Ex. | from example | [ppm] | [nm] | [nm] | YI |
| 23 | 2 | 2000 | 384 | 386 | 1.7 |
| 24 | 2 | 500 | 376 | 380 | 0.7 |
| 25 | 2 | 200 | — | 367 | −0.2 |
| 26 | 24 | 2000 | 386 | 389 | 3.9 |
| 27 | 24 | 500 | 372 | 377 | −0.01 |
| 28 | 24 | 200 | — | 373 | 0.09 |
| 29 | 25 | 500 | 391 | 394 | 1.5 |
| 30 | 6 | 500 | 384 | 391 | 2.1 |
| 31 | 8 | 2000 | 380 | 384 | 2.5 |
| 32 | 23 | 2000 | 387 | 389 | 3.5 |
| 33 | 26 | 2000 | 384 | 386 | 2.1 |

| | | | | |
|---|---|---|---|---|
| 34 | No UV absorber (PET alone) | 318 | 319 | −0.6 |

*Maximum wavelength with a transmittance <10% or <20% respectively.

TABLE 2b

| | | | Wavelength | Wavelength | |
|---|---|---|---|---|---|
| | Compound | Concentration | 10%* | 20%* | |
| Ex. | from example | [ppm] | [nm] | [nm] | YI |
| C3 | 3 | 2000 | 435 | 437 | 37.98 |
| C4 | 6 | 2000 | 395 | 400 | 10.76 |

*Maximum wavelength with a transmittance <10% or <20% respectively.

The pyridinedione derivatives and also the comparison compounds C1 and C2 were all readily incorporable into the PET film and the major part of the damaging UV radiation was filtered out of the spectrum. The comparison compounds C1 and C2, however, do not exhibit acceptable YI values.

### Example 35

### Incorporation of a Stabilizer Mixture into PET

A mixture of one part by weight of the pyridinedione derivative from example 2 and four parts by weight of the UV absorber Uvinul® 3030 (manufacturer: BASF Aktiengesellschaft) was incorporated in accordance with the method described above, in a concentration of 2500 ppm (by weight), into PET. The results are shown in Table 3.

TABLE 3

| | | Concen- | Wavelength | Wavelength | |
|---|---|---|---|---|---|
| | | tration | 10%* | 20%* | |
| Ex. | Stabilizer mixture | [ppm] | [nm] | [nm] | YI |
| 35 | 1 part compound Ex. 2 | 2500 | 376 | 379 | −0.7 |
| | 4 parts Uvinul ® 3030 | | | | |

*Maximum wavelength with a transmittance <10% or <20% respectively.

### Examples 36 and 37

### Comparison of the Pyridinedione Derivatives from Example 2 with Commercial UV Absorbers

The commercially available UV absorbers Tinuvin® 1577 (Ciba Specialty Chemicals) and Cyasorb® UV 24 (Cytec Industries) were each incorporated in a concentration of 2000 ppm (by weight) into PET, in the same way as described above. The results are reproduced in Table 4.

TABLE 4

| | | Concen- | Wavelength | Wavelength | |
|---|---|---|---|---|---|
| | | tration | 10%* | 20%* | |
| Ex. | UV Absorber | [ppm] | [nm] | [nm] | YI |
| 23 | Compound from | 2000 | 384 | 386 | 1.7 |
| | Ex. 2 | | | | |
| 36 | Tinuvin ® 1577 | 2000 | 372 | 376 | 1.6 |
| 37 | Cyasorb ® UV 24 | 2000 | 384 | 390 | 4.9 |

*Maximum wavelength with a transmittance <10% or <20% respectively.

Example 36 shows that the pyridinedione derivative from Example 2, with a similar yellowness index in the longer-wave UV region, possesses a higher absorption than Tinuvin® 1577.

Example 37 shows that the pyridinedione derivative from Example 2, with a similar UV absorption profile, exhibits much lower yellowing than Cyasorb® UV 24.

### Example 38

### Light Exposure of Samples

The PET film from Example 23 was exposed in accordance with DIN 54004 and the absorption profile was determined. The data are shown in Table 5.

TABLE 5

| Time | Wavelength 10%* | Wavelength 20%* |
|---|---|---|
| [h] | [nm] | [nm] |
| 0 | 384 | 386 |
| 100 | 384 | 386 |
| 200 | 384 | 386 |
| 400 | 383 | 385 |
| 600 | 382 | 385 |
| 1000 | 382 | 384 |

From Table 5 it is apparent that the UV-absorbing action of the pyridinedione derivative from Example 2 does not decrease significantly even on prolonged exposure.

### Example 39

### UV Barrier Properties of PET Films

In order to test the barrier properties of the additized films the light resistance of dyes of known photostability was tested behind the PET film from Example 23 (labeled in Table 6 as “a”—additized) and behind a non-additized film (identified in Table 6 as “n-a”—non-additized). A number of wool samples dyed with the blue dyes of European light fastness grades 4, 5 and 6 (EN ISO 105-B01) were exposed in accordance with DIN 54004, the wool samples being covered with the PET films. Measurements were made of the color spacing ΔE of the exposed pieces of wool in comparison to their unexposed counterparts. A low color spacing ΔE denotes minimal damage to the dye. Table 6 reproduces the color changes of the light fastness grades.

TABLE 6

| | | Light fastness | | | Light fastness | | | Light fastness | | |
|---|---|---|---|---|---|---|---|---|---|---|
| | Time | grade 4 | | | grade 5 | | | grade 6 | | |
| Film | (h) | 0 | 400 | 1200 | 0 | 400 | 1200 | 0 | 400 | 1200 |
| a | ΔE | 0.00 | 5.31 | 15.00 | 0.00 | 4.85 | 11.48 | 0.00 | 2.32 | 4.07 |
| n-a | ΔE | 0.00 | 14.06 | 30.61 | 0.00 | 11.84 | 22.89 | 0.00 | 5.64 | 13.28 |

The ΔE values after 0, 400 and 1200 h of exposure show that the color change of the blue dyes under consideration is markedly lower if a pyridinedione derivative is used in accordance with the invention.

1. A method of using a pyridinedione derivative of general formula I 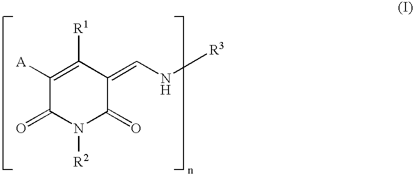
or a tautomer thereof
in which
R¹ is hydrogen, or a heteroatom-comprising alkyl, alkenyl or alkynyl or optionally substituted cycloalkyl, cycloalkenyl, heterocycloalkyl, aryl or heteroaryl,
R² independently of R¹ has the definition of R¹ or NR⁴R¹,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹,
n denotes values of 1, 2, 3 or 4
and
R³ if n is 1:
is hydrogen, or a heteroatom-comprising alkyl, alkenyl or alkynyl or optionally substituted cycloalkyl, cycloalkenyl or heterocycloalkyl,
if n is not 1:
is an n-valent aliphatic or cycloaliphatic radical which optionally comprise heteroatoms,
to protect organic material from the damaging effects of light.

2. The method according to claim 1, wherein
R¹ is hydrogen,
C₁-C₃₀ alkyl which is optionally interrupted by one or more nonadjacent groups selected from —O—, —S—, —NZ¹—, —CO— and —SO₂— and/or optionally substituted by identical or different radicals selected from the group consisting of cyano, amino, hydroxyl, halogen, carboxyl, C₁-C₁₈ alkoxycarbonyl, C₁-C₁₈ alkanoyloxy, aryl, heterocycloalkyl and heteroaryl, with the aryl, heterocycloalkyl, and heteroaryl groups being unsubstituted or carrying one or more substitutents selected independently of one another from the group consisting of C₁-C₁₈ alkyl and C₁-C₆ alkoxy,
C₅-C₈ cycloalkyl or 5- to 8-membered heterocycloalkyl, which are unsubstituted or carry one or more C₁-C₆ alkyl groups,
or
aryl or heteroaryl which are unsubstituted or carry one or more radicals selected independently of one another from the group consisting of C₁-C₁₈ alkyl, C₁-C₆ alkoxy, cyano, CONZ²Z³, CO₂Z⁴,
R² independently of R¹ has the definition of R¹ or NR⁴R⁵,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R₁,
n denotes values of 1, 2, 3 or 4
and
R³ if n is 1:
is hydrogen,
C₁-C₃₀ alkyl which is optionally interrupted by one or more nonadjacent groups selected from —O—, —S—, —NZ⁵—, —CO— and —SO₂— and/or optionally substituted by identical or different radicals selected from the group consisting of cyano, amino, hydroxyl, halogen, carboxyl, C₁-C₁₈ alkoxycarbonyl, C₁-C₁₈ alkanoyloxy, aryl, heterocycloalkyl and heteroaryl, with the aryl, heterocycloalkyl, and heteroaryl groups being unsubstituted or carrying one or more substitutents selected independently of one another from the group consisting of C₁-C₁₈ alkyl and C₁-C₆ alkoxy,
or
C₅-C₈ cycloalkyl or 5- to 8-membered heterocycloalkyl, which are unsubstituted or carry one or more C₁-C₆ alkyl groups,
if n is not 1:
n-valent C₂-C₃₀ alkyl whose carbon chain is optionally interrupted by one or more nonadjacent groups selected from —O—, —S—, —NZ⁶—, —CO— and —SO₂—,
or
n-valent C₅-C₈ cycloalkyl or n-valent 5- to 8-membered heterocycloalkyl, which are unsubstituted or carry one or more C₁-C₆ alkyl groups,
Z¹, Z², Z³, Z⁴, Z⁵ and Z⁶ each independently of one another being hydrogen, C₁-C₁₈ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

3. The method according to claim 1, wherein
R¹ is hydrogen,
C₁-C₁₅ alkyl which is optionally interrupted by one or more nonadjacent groups selected from —O—, —NZ¹— and —CO— and/or optionally substituted by identical or different radicals selected from the group consisting of cyano, amino, hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₆ alkyl and C₁-C₆ alkoxy,
cyclopentyl or cyclohexyl, which are unsubstituted or carry one or more C₁-C₆ alkyl groups,
piperidyl which is unsubstituted or carries one or more C₁-C₆ alkyl groups
or
phenyl which is unsubstituted or carries one or more radicals selected independently of one another from the group consisting of C₁-C₆ alkyl, C₁-C₆ alkoxy, cyano, CONZ²Z³, CO₂Z⁴,
R² independently of R¹ has the definition of R¹ or NR⁴R⁴,
R⁴, R⁵ independently of one another and of R¹ have the definition of R¹ or COR⁶,
A is CN, COR⁷, COOR⁷ or CONR⁷R⁸,
R⁶, R⁷, R⁸ independently of one another and of R¹ have the definition of R¹,
n denotes values of 1, 2, 3 or 4
and
R³ if n is 1:
is hydrogen,
C₁-C₁₅ alkyl which is optionally interrupted by one or more nonadjacent groups selected from —O—, —NZ⁵—and —CO— and/or optionally substituted by identical or different radicals selected from the group consisting of cyano, amino, hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₆ alkyl and C₁-C₆ alkoxy,
cyclopentyl or cyclohexyl, which are unsubstituted or carry one or more C₁-C₆ alkyl groups,
or
piperidyl which is unsubstituted or carries one or more C₁-C₆ alkyl groups,
if n is not 1:
n-valent C₂-C₃₀ alkyl whose carbon chain is optionally interrupted by one or more nonadjacent groups selected from —O—, —NZ⁶—and —CO—,
or
n-valent C₅-C₈ cycloalkyl or n-valent 5- to 8-membered heterocycloalkyl, which are unsubstituted or carry one or more C₁-C₄ alkyl groups,
Z¹, Z², Z³, Z⁴, Z⁵ and Z⁶ each independently of one another being hydrogen, C₁-C₁₈ alkyl, aryl or heteroaryl, with aryl and heteroaryl each being unsubstituted or carrying one or more substitutents selected from the group consisting of C₁-C₆ alkyl, C₁-C₆ alkoxy, hydroxyl, carboxyl and cyano.

4. The method according to claim 1, wherein
R¹ is hydrogen,
C₁-C₈ alkyl which is optionally substituted with identical or different radicals selected from the group consisting of halogen and aryl, with the aryl group being unsubstituted or carrying one or more substitutents selected independently of one another from the group consisting of C₁-C₄ alkyl and C₁-C₄ alkoxy,
cyclopentyl or cyclohexyl, which are unsubstituted or carry one or more C₁-C₄ alkyl groups,
piperidyl which is unsubstituted or carries one or more C₁-C₄ alkyl groups
or
phenyl which is unsubstituted or carries one or more radicals selected independently of one another from the group consisting of C₁-C₄ alkyl, C₁-C₄ alkoxy and cyano,
R² independently of R¹ has the definition of R¹ or NR⁴R⁵,
R⁴, R⁵ independently of one another and of R¹ have the definition of R¹ or COR⁶,
A is CN,
R⁶ independently of R¹ has the definition of R¹,
n denotes values of 1, 2, 3 or 4
and
R³ if n is 1:
is hydrogen,
C₁-C₈ alkyl which is optionally interrupted by one or more nonadjacent groups selected from —O—, —NZ⁵—and —CO— and/or optionally substituted by identical or different radicals selected from the group consisting of hydroxyl, halogen, carboxyl, C₁-C₆ alkoxycarbonyl, C₁-C₆ alkanoyloxy and aryl, with the aryl group being unsubstituted or carrying one or more substitutents selected independently of one another from the group consisting of C₁-C₄ alkyl and C₁-C₄ alkoxy,
cyclopentyl or cyclohexyl, which are unsubstituted or carry one or more C₁-C₄ alkyl groups,
or
piperidyl which is unsubstituted or carries one or more C₁-C₄ alkyl groups,
if n is not 1:
n-valent C₂-C₁₂ alkyl whose carbon chain is optionally interrupted by one or more nonadjacent groups selected from —O— and —NZ⁶—,
n-valent cyclopentyl or n-valent cyclohexyl, which are unsubstituted or carry one or more C₁-C₄ alkyl groups,
or
n-valent 5- to 8-membered heterocycloalkyl which is unsubstituted or carries one or more C₁-C₄ alkyl groups,
Z⁵ and Z⁶ being hydrogen or C₁-C₆ alkyl.

5. The method according to claim 1, wherein
R¹ is hydrogen,
C₁-C₄ alkyl, partly fluorinated or perfluorinated C₁-C₄ alkyl
or
phenyl which is unsubstituted or carries one or more radicals selected independently of one another from the group consisting of C₁-C₄ alkyl, C₁-C₄ alkoxy and cyano,
R² independently of R¹ has the definition of R¹ or NR⁴R¹,
R⁴, R⁵ independently of one another and of R¹ have the definition of R¹ or COR⁶,
A is CN,
R⁶ independently of R¹ has the definition of R¹,
n denotes values of 1, 2, 3 or 4
and
R³ if n is 1:
is hydrogen,
C₁-C₈ alkyl which is optionally interrupted by one group —O— and/or optionally substituted by identical or different radicals selected from the group consisting of hydroxyl, fluoro, carboxyl, C₁-C₄ alkoxycarbonyl, C₁-C₄ alkanoyloxy and aryl, with the aryl group being unsubstituted or carrying one or more substitutents selected independently of one another from the group consisting of C₁-C₄ alkyl and C₁-C₄ alkoxy, cyclopentyl or cyclohexyl, which are unsubstituted or carry one or more C₁-C₄ alkyl groups,
or
piperidyl which is unsubstituted or carries one or more C₁-C₄ alkyl groups,
if n is not 1:
n-valent C₂-C₁₂ alkyl, n-valent cyclopentyl, n-valent cyclohexyl or n-valent piperidyl, which is unsubstituted or carries one or more C₁-C₄ alkyl groups.

6. The method of claim 1, wherein the organic material for protection is selected from the group consisting of plastics, polymer dispersions, paints, photographic emulsions, photographic layers, paper, human or animal skin, human or animal hair, cosmetic products, pharmaceutical products, cleaning products, and foods.

7. The method according to claim 6 to protect plastics.

8. The method of using at least one pyridinedione derivative of general formula I as defined in claim 1 for preparing a layer which absorbs ultraviolet light.

9. The method according to claim 8, wherein the layer is composed of a thermoplastic polymer.

10. The method of claim 1, wherein the organic material comprises at least one pyridinedione derivative of general formula I in an amount of from 0.01 to 10% by weight, based on the total weight of the material.

11. The method of claim 1 which includes the addition of at least one further light stabilizer which has at least one absorption maximum in the wavelength range from 280 to 320 nm.

12. A composition comprising at least one pyridinedione derivative of general formula I as defined in claim 1, in an amount which provides protection from the damaging effects of light, and at least one organic material.

13. A pyridinedione derivative of general formula I or a tautomer thereof according to claim 1.
